(19) United States
(12) Patent Application Publication
(10) Pub. No.: US 2005/0282199 A1
(43) Pub. Date: Dec. 22, 2005

(54) Method to predict prostate cancer

(76) Inventors: Kevin M. Slawin, Houston, TX (US); Michael Kattan, Cleveland Heights, OH (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 11/126,945

(22) Filed: May 11, 2005

Related U.S. Application Data
(60) Provisional application No. 60/569,805, filed on May 11, 2004.

(51) Int. Cl. C12Q 1/68; G01N 33/574; G06F 19/00; G01N 33/48; G01N 33/50

(52) U.S. Cl. 435/6; 435/7.23; 702/19

(57) ABSTRACT

A method for predicting the probability or risk of prostate cancer is provided.

### CROSS-REFERENCE TO RELATED APPLICATIONS

This application claims the benefit of the filing date of U.S. application Ser. No. 60/569,805, filed May 11, 2004, the disclosure of which is incorporated by reference herein.

### BACKGROUND

Prostate cancer is the most commonly diagnosed cancer and the second leading cause of cancer death for men in the United States. In 1999, an estimated 179,300 men were diagnosed with prostate cancer and 37,000 died of this disease. Despite the identification of several new potential biomarkers for prostate cancer (e.g., p53, p21, p27, and E-cadherin), prostate specific antigen (PSA) and the histologic Gleason score have remained the most commonly used predictors of prostate cancer biology. In fact, the widespread use of PSA-based screening has dramatically increased the number of men diagnosed and treated for clinically localized prostate cancer over the past decade. Concomitantly the incidence of clinical metastatic disease at the time of initial diagnosis has dropped considerably, in concert with an overall decrease in prostate cancer mortality (Merill et al., 2000).

Even given the significant rate of long-term cancer control afforded patients with clinically localized prostate cancer treated with radical prostatectomy or radiation therapy, approximately 30% of these patients will fail treatment, as evidenced by a detectable or rising PSA, which often is due to early dissemination of microscopic metastatic disease prior to primary therapy (Pound et al., 1997). Conventional staging modalities such as bone scan, CT scan, and MRI have a limited role in staging patients with clinically localized prostate cancer, because of their poor performance in detecting early, low-volume metastases (Oesterling et al., 1993; Engeler et al., 1992). Pre-operative nomograms that consider established markers such as PSA, clinical stage, and biopsy Gleason score can provide an estimate of the risk of nodal metastasis or disease recurrence, but are still imperfect for determining the pathological stage or prognosis in individual patients (Partin et al., 1997; Kattan et al., 1998). Improved pre-operative identification of patients with occult metastatic disease, who have a high probability of developing disease progression despite effective local therapy, would be helpful in sparing men from the morbidity of a radical prostatectomy or radiation therapy that would be ineffective or for selecting patients best suited for clinical trials of neoadjuvant or adjuvant therapy.

Recently, there has been a realization that pre-treatment PSA levels, the primary predictive parameter in the majority of tools to predict recurrence, may reflect primarily the presence of benign prostatic hyperplasia (BPH) rather than prostate cancer. Stamey et al. (2001) reported that for patients with a PSA level of ≦9 ng/mL, PSA poorly reflected the risk of progression after radical prostatectomy but was significantly correlated with the overall volume of the radical prostatectomy specimen, a direct reflection of the degree of BPH present. Several have failed to detect an independent predictive value for pre-operative PSA for disease progression in studies that have included more modern cohorts of patients with clinically localized prostate cancer undergoing radical prostatectomy who had lower median PSA levels than patients in most older studies.

While a number of molecules other than PSA are associated with prostate cancer, it is unclear whether any of these molecules, or which combinations of molecules, are useful to predict disease or disease outcome. Therefore, there is an imminent need for methods and nomograms that include markers that are specifically associated with disease or significant disease for improved prediction for patients with prostate-related disorders.

### SUMMARY OF THE INVENTION

The invention provides methods, apparatus and nomograms to predict the probability of prostate cancer and/or the probability of significant prostate cancer. “Significant prostate cancer” means more than one positive core, e.g., on extended biopsy (i.e., a biopsy with 10 or more cores), a Gleason score greater than 6, and/or a total cancer length of 3 mm or greater. The methods employ values or scores obtained from data that may include clinical data and/or data from physiological fluid sample(s) such as a protein found in the blood, to predict patient outcome, e.g., the risk or probability of prostate cancer. As used herein, a sample of “physiological fluid” includes, but is not limited to, a sample of blood, plasma, serum, seminal fluid, urine, saliva, sputum, semen, pleural effusions, bladder washes, bronchioalveolar lavages, cerebrospinal fluid and the like. In one embodiment, the methods employ values or scores for one or more factors including age, race, DRE, prostate volume, TZ volume, BPSA level (including concentration or amount), hK2 level (including concentration or amount), PSA level (including concentration or amount), free (non-complexed) PSA level (including concentration or amount), proPSA level (including concentration or amount), and/or other markers, to predict patient outcome. As used herein, “prostate volume” (PV) refers to size and weight of the prostate. As used herein, “PSA” refers to prostate-specific antigen. PSA is a protein produced by the prostate. An increased amount of PSA in the blood is linked to men who have prostate cancer, benign prostatic hyperplasia or an infection of the prostate gland. A blood sample is measured in an assay and the amount of PSA is reported as ng/ml. As used herein, “BPSA” or “benign PSA” refers to a specific molecular form of free prostate-specific antigen that is found predominantly in the transition zone of patients with nodular benign prostatic hyperplasia (Mikolajczyk et al., 2000; U.S. Pat. No. 6,482,599), but is also present in the serum. As used herein, “proPSA” refers to the form of PSA that in normal prostate glands is secreted into the glandular lumen where seven amino acids are cleaved to create active PSA. There are several isoforms of proPSA (i.e., −2, −4 and −7 proPSA). As used herein, “free PSA” (fPSA) refers to the various proPSA isoforms, intact free PSA and BPSA. Serum PSA that is measurable by current clinical immunoassays exists primarily as either the free “noncomplexed” form or as a complex with ACT (β₁-antichymotrypsin; Lilja et al., 1991; Stenman et al., 1991). As used herein, “intact, non-complexed PSA” refers to the free noncomplexed form of PSA described above.

In one embodiment, the invention provides a method to determine the risk of prostate cancer, e.g., the probability that a biopsy, such as an extended, e.g., at least 10 core, biopsy, detects prostate cancer, in a patient. The method includes providing a value for one or more of the following factors in a patient: age, race, DRE, PSA level, free PSA level, BPSA level, and/or proPSA level; and correlating the one or more values with the risk of prostate cancer, such as significant prostate cancer, in the patient. In one embodiment, two or more of the factor values are employed to predict the risk of prostate cancer. In another embodiment, three or more, e.g., four, five, six, or seven of the factor values are employed to predict the risk of prostate cancer. Also provided is a method for predicting the probability of prostate cancer in a patient. The method includes correlating a set of values for factors of a patient to a functional representation of a set of factors determined for each of a plurality of persons previously diagnosed with prostate cancer, so as to yield a value for total points for the patient. The set of factors includes at least one of age, race, DRE, PSA level, free PSA level, BPSA level, and/or proPSA level, and the functional representation includes a scale for each of age, race, DRE, PSA level, free PSA level, BPSA level, and/or proPSA level, a points scale, a total points scale, and a predictor scale. The scales for age, race, DRE, PSA level, free PSA level, BPSA level, and/or proPSA level, each have values on the scales which can be correlated with values on the points scale, and the total points scale has values which may be correlated with values on the predictor scale. The value on the total points scale for the patient is correlated with a value on the predictor scale to predict the quantitative probability of prostate cancer in the patient.

Also provided is an apparatus. The apparatus includes a data input means, for input of information for one or more factors from a patient including age, race, DRE, PSA level, free PSA level, BPSA level, and/or proPSA level; a processor, executing a software for analysis of the information, wherein the software analyzes the information and provides the risk of prostate cancer in the patient.

Further provided is an apparatus for predicting a probability of prostate cancer. The apparatus includes a correlation of a set of factors for each of a plurality of persons previously diagnosed with prostate cancer with the incidence of prostate cancer for each person of the plurality of persons. The set of factors includes one or more of age, race, DRE, PSA level, free PSA level, BPSA level, and/or proPSA level. The apparatus includes a means for comparing an identical set of factors determined from a patient to the correlation to predict the quantitative probability of prostate cancer and/or significant prostate cancer in the patient.

The invention also provides a nomogram for the graphic representation of the risk or a quantitative probability of prostate cancer in a patient. The nomogram includes a plurality of scales and a solid support. The plurality of scales is disposed on the support and includes a scale for one or more factors including age, race, DRE, PSA level, free PSA level, BPSA level, and/or proPSA level, a points scale, a total points scale and a predictor scale. The scales for age, race, DRE, PSA level, free PSA level, BPSA level, and/or proPSA level each has values on the scales. The scales for age, race, DRE, PSA level, free PSA level, BPSA level, and/or proPSA level are disposed on the solid support with respect to the points scale so that each of the values on age, race, DRE, PSA level, free PSA level, BPSA level, and/or proPSA level can be correlated with values on the points scale. The total points scale has values on the total points scale, and the total points scale is disposed on the solid support with respect to the predictor scale so that the values on the total points scale may be correlated with values on the predictor scale, such that the values on the points scale correlating with the patient's age, race, DRE, PSA level, free PSA level, BPSA level, and/or proPSA level can be added together to yield a total points value. The total points value can be correlated with the predictor scale to predict the risk or quantitative probability of prostate cancer.

Also provided is an apparatus for predicting prostate cancer in a patient. The apparatus comprises: a scale for one or more of age, race, DRE, PSA level, free PSA level, BPSA level, and/or proPSA level, a points scale, a total points scale and a predictor scale. The scales for age, race, DRE, PSA level, free PSA level, BPSA level, and/or proPSA level each has values on the scales. The scales for age, race, DRE, PSA level, free PSA level, BPSA level, and/or proPSA level are disposed so that each of the values on age, race, DRE, PSA level, free PSA level, BPSA level, and/or proPSA level can be correlated with values on the points scale. The total points scale has values on the total points scale, and the total points scale is disposed on the solid support with respect to the predictor scale so that the values on the total points scale may be correlated with values on the predictor scale, such that the values on the points scale correlating with the patient's age, race, DRE, PSA level, free PSA level, BPSA level, and/or proPSA level can be added together to yield a total points value. The total points value can be correlated with the predictor scale to predict the probability or risk of prostate cancer.

The invention further provides a method to determine the risk or quantitative probability of a prostate cancer in a patient. The method includes inputting information to a data input means, wherein the information comprises values for one or more factors from a patient including age, race, DRE, PSA level, free PSA level, BPSA level, and/or proPSA level, executing a software for analysis of the information; and analyzing the information so as to provide the risk or quantitative probability of prostate cancer in the patient.

The invention also provides a method for predicting prostate cancer in a patient. The method includes correlating a set of values for factors of a patient to a functional representation of a set of factors determined for each of a plurality of persons previously diagnosed with prostate cancer, so as to yield a value for total points for the patient. The set of factors includes at least one of age, race, DRE, PSA level, free PSA level, BPSA level, and/or proPSA level. The functional representation includes a scale for each of age, race, DRE, PSA level, free PSA level, BPSA level, and/or proPSA level, a points scale, a total points scale, and a predictor scale. The scales for age, race, DRE, PSA level, free PSA level, BPSA level, and/or proPSA level, each have values on the scales which can be correlated with values on the points scale, and the total points scale has values which may be correlated with values on the predictor scale. The value on the total points scale for the patient with a value on the predictor scale to predict the quantitative probability of prostate cancer in the patient.

The invention also provides methods, apparatus and nomograms to predict the status, e.g., disease-free status, of a prostate cancer patient after therapy, e.g., after radical prostatectomy, external beam radiation therapy, brachytherapy, or other localized therapies for prostate cancer, e.g., cryotherapy. The methods employ values or scores from biopsies, such as a 12 core biopsy set, prostatectomy final pathology, and/or other markers, e.g., markers present in a physiological fluid sample such as a protein found in the blood, to predict patient outcome. The biopsy or physiological fluid, e.g., blood sample, may be obtained from the patient prior to and/or after therapy for prostate cancer. When the sample is collected “after” therapy, it may be collected at times up to about 5 to 6 months, e.g., about 1, 2, 3, 4, or more months, e.g., 7, 8, 9, 10 or 11 months, after therapy, including from about 1, 2, 3, 4 or 5 days after therapy, up to about 1, 2, 3, 4, 5, or 6 weeks after therapy. In other embodiments, the sample may be collected years after therapy such as about 1, 2, 3, 4, 5, 6 or 7 years after therapy. In one embodiment, the sample is collected after therapy, for instance, at a time when PSA levels or amount are monitored or when PSA levels or amounts are rising over time.

In one embodiment, the invention includes correlating the value or score from various markers, such as protein markers, biopsy data, e.g., 12 core systematic biopsy data, and/or optionally prostatectomy final pathology, for example, in a nomogram, to predict, for instance, patient outcome, progression, risk of organ-confined disease, extracapsular extension, seminal vesicle invasion, and/or lymph node involvement. In another embodiment, the invention includes correlating the value or score from various markers, such as protein markers found in blood, biopsy data, e.g., 12 core systematic biopsy data, and/or optionally prostatectomy final pathology, from a patient with metastatic disease, either hormone sensitive or hormone refractory metastatic disease, to predict the aggressiveness of the disease and/or time to death.

For instance, the methods, apparatus or nomograms may be employed prior to localized therapy for prostate cancer, e.g., to predict risk of progression or predict organ-confined disease, after therapy for prostate cancer such as in patients with PSA recurrence, e.g., to predict aggressiveness of recurrence, time to metastasis and/or time to death, or, in patients with metastatic disease or hormone refractory metastatic disease, e.g., to predict the aggressiveness of disease and/or time to death.

As described herein, 178 patients with no prior history of prostate biopsy, who had prostate cancer diagnosed during an initial systematic 12-core (S12C) biopsy, and who subsequently underwent radical prostatectomy were studied. The comparison groups included the subset of the six standard sextant cores (S6C), the set of six laterally directed cores (L6C), and the complete 12 core set (S12C) that included both the six standard sextant and six laterally directed cores. Biopsy Gleason score, number of positive cores, total length of cancer, and percent of tumor in the biopsy sets were examined for their ability to predict extracapsular extension, total tumor volume, and pathologic Gleason score. Analyses were performed using Spearman's rho correlation and multivariable regression analyses. In univariable analyses, the S12C correlated most strongly with the presence of extracapsular extension and total tumor volume, compared to either the S6C or the L6C. In multivariable analyses, both the S6C and L6C were independent predictors of post-prostatectomy pathologic parameters. Thus, the addition of 6 systematically obtained, laterally directed cores to the standard sextant biopsy significantly improves the ability to predict pathologic features by a statistically and prognostically or significant margin. Pre-operative nomograms that utilize data from a full complement of 12 systematic sextant and laterally directed biopsy cores can thus improve performance in predicting post-prostatectomy pathology (e.g., indolent cancer or the presence of extracapsular extension). In one embodiment, Gleason score, number of positive cores, number of positive contiguous cores, total cancer length, total length of cancer in contiguous cores, and/or percent tumor involvement are correlated to post-prostatectomy pathology. Moreover, in patients with a negative S12C, initial digital rectal exam status and/or the presence of prostatic intraepithelial neoplasia was found to an indication to rebiopsy, e.g., to perform a second S12C.

To better counsel men diagnosed with prostate cancer, a statistical model that accurately predicts the presence and extent of cancer based on clinical variables (serum PSA, clinical stage, prostate biopsy Gleason grade, and ultrasound volume), and variables derived from the analysis of systematic biopsies, was developed. The analysis included 1,022 patients diagnosed through systematic needle biopsy with clinical stages Tlc to T3 NO or NX, and MO or MX prostate cancer who were treated solely with radical prostatectomy. Overall, 105 (10%) of the patients had indolent cancer. The nomogram predicted the presence of an indolent cancer with discrimination for various models ranging from 0.82 to 0.90. Thus, nomograms incorporating pre-treatment variables (clinical stage, Gleason grade, PSA, and/or the amount of cancer in a systematic biopsy specimen) can predict the probability that a man with prostate cancer has an indolent tumor.

The invention provides a method to determine the risk of indolent cancer, or the risk of posterolateral extracapsular extension of prostate cancer, in a patient prior to therapy for prostate cancer. The method comprises correlating one or more of pre-treatment PSA, TGF-β₁, IGF BP-2, IL-6, IL6sR, IGF BP-3, UPA, UPAR, VEGF and/or sVCAM; clinical stage; biopsy Gleason scores, number of positive cores, total length of cancer, and/or the percent of tumor in a 12 core set of prostate biopsies from the patient, with the risk of indolent cancer and/or posterolateral extracapsular extension. Such information can enhance treatment decisions.

Hence, the invention also provides a method to predict the presence of indolent prostate tumors. In one embodiment, the method includes correlating a set of factors for a radical prostatectomy patient to a functional representation of a set of factors determined for each of a plurality of patients previously diagnosed with prostate cancer and having been treated by radical prostatectomy, e.g., pre-treatment PSA level, clinical stage, Gleason grade, size of cancerous tissue, size of non-cancerous tissue, and/or ultrasound or transrectal ultrasound (U/S) volume. Then the value for each factor for the patient is correlated to a value on a predictor scale to predict the presence of indolent prostate tumors in the patient.

To develop a nomogram to predict the side of extracapsular extension, 763 patients with clinical stage Tlc-T3 prostate cancer who were diagnosed with a systematic biopsy and were subsequently treated with radical prostatectomy were studied. The variables studied included an abnormality on DRE, the worst Gleason score, number of cores with cancer, percent cancer in a biopsy specimen on each side, and serum PSA level. The area under the curve of DRE, biopsy Gleason sum and PSA in predicting the side of ECE was 0.648 and 0.627, respectively, and was 0.763 when these parameters were combined. Further, this was enhanced by adding the information of systematic biopsy with the highest value of 0.787 with a percent cancer. A nomogram incorporating pre-treatment variables on each side of the prostate can thus provide accurate prediction of the side of extracapsular extention in prostate biopsy specimens.

The invention provides a method to predict the side of extracapsular extension in radical prostatectomy specimens. In one embodiment, the method includes correlating a set of factors for a radical prostatectomy patient to a functional representation of a set of factors determined for each of a plurality of patients previously diagnosed with prostate cancer and having been treated by radical prostatectomy, e.g., factors including pre-treatment PSA and, in a biopsy, worst Gleason score, number of cores with cancer, and/or percent cancer in a biopsy specimen on each side. Then the value for each factor for the patient is correlated to a value on a predictor scale to predict the side of extracapsular extension in the prostate of a patient.

To develop a nomogram to improve the accuracy of predicting the freedom from PSA progression after salvage radiotherapy (XRT) for biochemical recurrence following prostatectomy, pre- and post-prostatectomy clinical-pathological data and disease follow-up for 303 patients receiving salvage XRT was modeled using Cox proportional hazards regression analysis. It was found that pre-XRT PSA and Gleason grade were the strongest predictors of treatment success. Thus, a minority of patients may derive a durable benefit from salvage radiotherapy for suspected local recurrence. Accordingly, a nomogram can aid in identifying the most appropriate patients to receive salvage XRT.

Hence, also provided is a method to predict the outcome of salvage radiotherapy after biochemical recurrence in prostate cancer patients treated with radical prostatectomy. In one embodiment, the method includes correlating a set of factors for a radical prostatectomy patient to a functional representation of a set of factors determined for each of a plurality of patients previously diagnosed with prostate cancer and having been treated by radical prostatectomy, e.g., pre-treatment PSA level, pre-salvage radiotherapy PSA level, Gleason sum, pathological stage, pre-salvage radiotherapy PSA doubling time, positive surgical margins, time to biochemical recurrence, and pre-salvage radiotherapy neoadjuvant hormone therapy. Then the value for each factor for the patient is correlated to a value on a predictor scale to predict the outcome of salvage radiotherapy after biochemical recurrence in prostate cancer patients treated with radical prostatectomy.

The invention also includes the use of nomograms to predict time to death in patients with advanced prostate cancer. In one embodiment, the nomogram predicts time to death in patients with hormone sensitive metastatic prostate cancer. In another embodiment, the nomogram predicts the time to death in patients with hormone refractory prostate cancer. Nomograms may include markers present in physiological fluids, e.g., TGF-β₁, UPA, VEGF, and the like, as well as standard clinical parameters, including those in Smaletz et al. (2002), the disclosure of which is specifically incorporated by reference herein. Moreover, the presence of certain markers after primary therapy, e.g., PSA recurrence after primary therapy, may be employed to predict the aggressiveness of recurrence, the time to metastases, and/or time to death.

To determine whether transition zone volume (TZV) and total prostate volume (TPV) are independent predictors of PSA, results from 560 men who underwent a systematic 12-core biopsy performed under ultrasound guidance were analyzed. When controlling for race, age and biopsy status using linear regression, TZV and TPV are each separately significant predictors of PSA (P<0.0001 each).

### BRIEF DESCRIPTION OF THE FIGURES

FIG. 1. Diagram of posterior view of prostate with systematic 12-core biopsy locations marked. Coronal view. Inner circle represents prostatic transition zone. Inner ellipsoid represents transitional zone. X, sextant locations; O, laterally directed locations; ML, midline; B, base; M, mid; A, apex. The circle indicates the anterioposterior and lateral extant of the translational zone in a patient with moderate BPH.

FIG. 2. Nomogram to predict the side of extracapsular extension in radical prostatectomy specimens. BXTGS=biopsy total Gleason score; CSTAGE=clinical stage; PERCA=percent cancer in a biopsy specimen.

FIG. 3. Nomogram to predict progression-free probability post-radiotherapy.

FIG. 4. Nomogram to predict the presence of indolent prostate tumors.

FIGS. 5A-B. Plasma UPA and UPAR levels in various patient populations.

FIG. 6. Flow chart.

FIG. 7. Nomogram for patients with hormone refractory disease.

FIGS. 8A-D. A) Nomogram to predict prostate cancer. B) Nomogram to predict significant prostate cancer. C) and D) Exemplary results using the two nomograms.

### DETAILED DESCRIPTION OF THE INVENTION

The invention includes a method to predict the probability of prostate cancer and/or probability of significant prostate cancer in a patient. The invention also includes a method to predict organ confined (local) prostate disease status, the potential for progression of prostate cancer following primary therapy, e.g., the presence of occult metastases, the side and extent of extracapsular extension of prostate cancer, the risk of extracapsular extension in the area of the neurovascular bundle (posterolaterally), and/or the presence of indolent prostate tumor in patients; the aggressiveness of disease, time to metastasis and/or time to death in patients with PSA recurrence; and the aggressiveness of disease and/or time to death in patients with metastases, e.g., those with or without hormone refractory disease. Specifically, the detection of pre- or post-operative TGF-β₁, IL-6, IL6sR, IGFBP-2, IGFBP-3, UPA, UPAR, VEGF, sVCAM, BPSA or PSA (including PSA and/or free PSA) levels alone, or in conjunction with parameters derived from a 10 or more, e.g., 12, core systemic biopsy of the prostate, final pathology, age, race, DRE or yet other markers for prostate cancer, may be useful in predicting, for example, prostate cancer, or organ-confined disease status or the potential for progression in patients with clinically localized prostate cancer. In one embodiment, the method is useful for evaluating patients at risk for recurrence of prostate cancer following primary therapy for prostate cancer.

Non-invasive prognostic assays are provided by the invention to detect and/or quantitate TGF-β₁, IL-6, IL6sR, IGFBP-2, IGFBP-3 UPA, UPAR, VEGF, sVCAM, BPSA or PSA levels in the body fluids of mammals, including humans. Thus, such an assay is useful in prognosis of prostate cancer. Moreover, such assays provide valuable means of monitoring the status of the prostate cancer. In addition to improving prognostication, knowledge of the disease status allows the attending physician to select the most appropriate therapy for the individual patient. For example, patients with a high likelihood of relapse can be treated rigorously. Because of the severe patient distress caused by the more aggressive therapy regimens as well as prostatectomy, it would be desirable to distinguish with a high degree of certainty those patients requiring aggressive therapies as well as those which will benefit from prostatectomy.

The body fluids that are of particular interest as physiological samples in assaying for TGF-β₁, IL-6, IL6sR, IGFBP-2, IGFBP-3, UPA, UPAR, VEGF, sVCAM, BPSA or PSA according to the methods of this invention include blood, blood serum, semen, saliva, sputum, urine, blood plasma, pleural effusions, bladder washes, bronchioalveolar lavages, and cerebrospinal fluid. Blood, serum and plasma are preferred, and plasma, such as platelet-poor plasma, are the more preferred samples for use in the methods of this invention.

Exemplary means for detecting and/or quantitating TGF-β₁, IL-6, IL6sR, IGFBP-2, IGFBP-3, UPA, UPAR, VEGF, sVCAM, BPSA or PSA levels in mammalian body fluids include affinity chromatography, Western blot analysis, immunoprecipitation analysis, and immunoassays, including ELISAs (enzyme-linked immunosorbent assays), RIA (radioimmunoassay), competitive EIA or dual antibody sandwich assays. In such immunoassays, the interpretation of the results is based on the assumption that the TGF-β₁, IL-6, IL6sR, IGFBP-2, IGFBP-3, UPA, UPAR, VEGF, sVCAM, BPSA or PSA binding agent, e.g., a TGF-β₁, IL-6, IL6sR, IGFBP-2, IGFBP-3, UPA, UPAR, VEGF, sVCAM, BPSA or PSA specific antibody, will not cross-react with other proteins and protein fragments present in the sample that are unrelated to TGF-β₁, IL-6, IL6sR, IGFBP-2, IGFBP-3, UPA, UPAR, VEGF, sVCAM, BPSA or PSA. Preferably, the method used to detect TGF-β₁, IL-6, IL6sR, IGFBP-2, IGFBP-3, UPA, UPAR, VEGF, sVCAM, BPSA or PSA levels employs at least one TGF-β₁, IL-6, IL6sR, IGFBP-2, IGFBP-3, UPA, UPAR, VEGF, sVCAM, BPSA or PSA specific binding molecule, e.g., an antibody or at least a portion of the ligand for any of those molecules. Immunoassays are a preferred means to detect TGF-β₁, IL-6, IL6sR, IGFBP-2, IGFBP-3, UPA, UPAR, VEGF, sVCAM, BPSA or PSA. Representative immunoassays involve the use of at least one monoclonal or polyclonal antibody to detect and/or quantitate TGF-β₁, IL-6, IL6sR, IGFBP-2, IGFBP-3, UPA, UPAR, VEGF, sVCAM, BPSA or PSA in the body fluids of mammals. The antibodies or other binding molecules employed in the assays may be labeled or unlabeled. Unlabeled antibodies may be employed in agglutination; labeled antibodies or other binding molecules may be employed in a wide variety of assays, employing a wide variety of labels.

Suitable detection means include the use of labels such as radionucleotides, enzymes, fluorescers, chemiluminescers, enzyme substrates or co-factors, enzyme inhibitors, particles, dyes and the like. Such labeled reagents may be used in a variety of well known assays. See for example, U.S. Pat. Nos. 3,766,162, 3,791,932, 3,817,837, and 4,233,402.

Still further, in, for example, a competitive assay format, labeled TGF-β₁, IL-6, IL6sR, IGFBP-2, IGFBP-3, UPA, UPAR, VEGF, sVCAM, BPSA or PSA peptides and/or polypeptides can be used to detect and/or quantitate TGF-β₁, IL-6, IL6sR, IGFBP-2, IGFBP-3, UPA, UPAR, VEGF, sVCAM, BPSA or PSA, respectively, in mammalian body fluids. Also, alternatively, as a replacement for the labeled peptides and/or polypeptides in such a representative competitive assay, labeled anti-idiotype antibodies that have been prepared against antibodies reactive with TGF-β₁, IL-6, IL6sR, IGFBP-2, IGFBP-3, UPA, UPAR, VEGF, sVCAM, BPSA or PSA can be used.

It can be appreciated that certain molecules such as TGF-β₁ may be present in various forms, e.g., latent and active, as well as fragments thereof, and that these various forms may be detected and/or quantitated by the methods of the invention if they contain one or more epitopes recognized by the respective binding agents. For example, in a sandwich assay where two antibodies are used as a capture and a detection antibody, respectively, if both epitopes recognized by those antibodies are present on at least one form of, for example, TGF-β₁, the form would be detected and/or quantitated according to such an immunoassay. Such forms which are detected and/or quantitated according to methods of this invention are indicative of the presence of the active form in the sample.

For example, TGF-β₁, IL-6, IL6sR, IGFBP-2, IGFBP-3, UPA, UPAR, VEGF, sVCAM, BPSA or PSA levels may be detected by an immunoassay such as a “sandwich” enzyme-linked immunoassay (see Dasch et al., 1990; Danielpour et al., 1989; Danielpour et al., 1990; Lucas et al., 1990; Thompson et al., 1989; and Flanders et al., 1989). A physiological fluid sample is contacted with at least one antibody specific for TGF-β₁, IL-6, IL6sR, IGFBP-2, IGFBP-3, UPA, UPAR, VEGF, sVCAM, BPSA or PSA to form a complex with said antibody and TGF-β₁, IL-6, IL6sR, IGFBP-2, IGFBP-3, UPA, UPAR, VEGF, sVCAM, BPSA or PSA. Then the amount of TGF-β₁ in the sample is measured by measuring the amount of complex formation. Representative of one type of ELISA test is a format wherein a solid surface, e.g., a microtiter plate, is coated with antibodies to TGF-β₁, IL-6, IL6sR, IGFBP-2, IGFBP-3, UPA, UPAR, VEGF, sVCAM, BPSA or PSA and a sample of a patient's plasma is added to a well on the microtiter plate. After a period of incubation permitting any antigen to bind to the antibodies, the plate is washed and another set of TGF-β₁, IL-6, IL6sR, IGFBP-2, IGFBP-3, UPA, UPAR, VEGF, sVCAM, BPSA or PSA antibodies, e.g., antibodies that are linked to a detectable molecule such as an enzyme, is added, incubated to allow a reaction to take place, and the plate is then rewashed. Thereafter, enzyme substrate is added to the microtiter plate and incubated for a period of time to allow the enzyme to catalyze the synthesis of a detectable product, and the product, e.g., the absorbance of the product, is measured.

It is also apparent to one skilled in the art that a combination of antibodies to TGF-β₁, IL-6, IL6sR, IGFBP-2, IGFBP-3, UPA, UPAR, VEGF, sVCAM, BPSA or PSA can be used to detect and/or quantitate the presence of TGF-β₁, IL-6, IL6sR, IGFBP-2, IGFBP-3, UPA, UPAR, VEGF, sVCAM, BPSA or PSA in the body fluids of patients. In one such embodiment, a competition immunoassay is used, wherein TGF-β₁, IL-6, IL6sR, IGFBP-2, IGFBP-3, UPA, UPAR, VEGF, sVCAM, BPSA or PSA is labeled, and a body fluid is added to compete the binding of the labeled TGF-β₁, IL-6, IL6sR, IGFBP-2, IGFBP-3, UPA, UPAR, VEGF, sVCAM, BPSA or PSA to antibodies specific for TGF-β₁, IL-6, IL6sR, IGFBP-2, IGFBP-3, UPA, UPAR, VEGF, sVCAM, BPSA or PSA. Such an assay could be used to detect and/or quantitate TGF-β₁ IL-6, IL6sR, IGFBP-2, IGFBP-3, UPA, UPAR, VEGF, sVCAM, BPSA or PSA.

Thus, once binding agents having suitable specificity have been prepared or are otherwise available, a wide variety of assay methods are available for determining the formation of specific complexes. Numerous competitive and non-competitive protein binding assays have been described in the scientific and patent literature and a large number of such assays are commercially available. Exemplary immunoassays which are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Methods to detect TGF-β₁ levels as well as TGF-β₁ binding molecules are well known to the art (see, e.g., U.S. Pat. Nos. 5,216,126, 5,229,495, 5,571,714, and 5,578,703; WO 91/08291; WO 93/09228; WO 93/09800; and WO 96/36349).

The methods of the invention may be employed with other measures of prostate cancer biology to better predict disease-free status or for staging. For example, the following clinical and pathological criteria may be used, e.g., age, race, DRE, clinical or pathological stage, PSA levels, Gleason values, e.g., primary Gleason grade, secondary Gleason grade, or Gleason sum (score) and/or core data, although the use of other criteria does not depart from the scope and spirit of the invention.

T0—No evidence of prostatic tumor.

T1—Clinically inapparent tumor, non-palpable nor visible by imaging.

T1a—Tumor is incidental histologic finding with three of fewer microscopic foci. Non-palpable, with 5% or less of TURP chips (trans-urethral resected prostate tissue) positive for cancer.

T1b—Tumor is incidental histologic finding with more than three microscopic foci. Non-palpable, with greater than 5% of TURP chips (trans-urethral resected prostate tissue) positive for cancer.

T1c—Tumor is non-palpable, and is found in one or both lobes by needle biopsy diagnosis.

T2—Tumor is confined within the prostate.

T2a—Tumor present clinically or grossly, limited to the prostate, tumor 1.5 cm or less in greatest dimension, with normal tissue on at least three sides. Palpable, half of 1 lobe or less.

T2b—Tumor present clinically or grossly, limited to the prostate, tumor more than 1.5 cm in greatest dimension, or in only one lobe. Palpable, greater than half of 1 lobe but not both lobes.

T2c—Tumor present clinically or grossly, limited to the prostate, tumor more than 1.5 cm in greatest dimension, and in both lobes. Palpable, involves both lobes.

T3—Tumor extends through the prostatic capsule.

T3a—Palpable tumor extends unilaterally into or beyond the prostatic capsule, but with no seminal vesicle or lymph node involvement. Palpable, unilateral capsular penetration.

T3b—Palpable tumor extends bilaterally into or beyond the prostatic capsule, but with no seminal vesicle or lymph node involvement. Palpable, bilateral capsular penetration.

T3c—Palpable tumor extends unilaterally and/or bilaterally beyond the prostatic capsule, with seminal vesicle and/or lymph node involvement. Palpable, seminal vesicle or lymph node involvement.

T4—Tumor is fixed or invades adjacent structures other than the seminal vesicles or lymph nodes.

T4a—Tumor invades any of: bladder neck, external sphincter, rectum.

T4b—Tumor invades levator muscles and/or is fixed to pelvic wall.

TABLE 1

Gleason grade
in biopsy†

| Primary | Secondary | No. patients (%) |
|---|---|---|
| 1-2 | 1-2 | 108 (11.0) |
| 1-2 | 3 | 158 (16.1) |
| 3 | 1-2 | 65 (6.6) |
| 3 | 3 | 340 (34.6) |
| 1-3 | 4-5 | 213 (21.7) |
| 4-5 | 1-5 | 99 (10.1) |

†Gleason grades 1-2 are well differentiated, 3 is moderately differentiated, 4-5 are poorly differentiated.

TABLE 2

| Pre-operative PSA‡ | No. patients (%) |
|---|---|
| 0.1-4.0 | 217 (22.1) |
| 4.1-10.0 | 472 (48.0) |
| 10.1-20.0 | 187 (19.0) |
| 20.1-100.0 | 107 (10.9) |

‡Median serum prostate-specific antigen (PSA) level for all patients. 6.8 ng/mL (range, 0.1-100.0 ng/mL); mean serum PSA level for all patients, 9.9 ng/mL (95% confidence interval = 9.24-10.54 ng/mL).

Exemplary Methods, Apparatus and Nomograms with Pre-Therapy Variables

The present invention provides methods, apparatus and nomograms to predict disease or disease recurrence using factors available prior to treatment, e.g., prior to surgery, to aid patients considering treatment such as radical prostatectomy to treat clinically localized prostate cancer, as well as to predict disease recurrence after salvage radiation therapy in prostate cancer patients, to predict extracapsular extension in prostate cancer patients, prostatic intraepithelial neoplasia in prostate cancer patients, and/or indolent cancer in prostate cancer patients. In one embodiment, a nomogram predicts the probability of disease using pretreatment, e.g., pre-operative, factors. The selected set of factors includes, but is not limited to, age, race, DRE, PSA level, free PSA level, BPSA level, and/or proPSA level. For example, a selected set of factors determined for each of a plurality of persons previously diagnosed with prostate cancer is correlated with the risk of prostate cancer for each person of the plurality of persons, so as to generate a functional representation of the correlation. An identical set of factors determined for the patient in matched to the functional representation so as to predict the risk of prostate cancer in that patient. Thus, the nomogram may be used in clinical decision making by the clinician and patient and may be used to identify patients at high risk of disease.

In one embodiment, a pre-operative nomogram predicts the probability of disease recurrence after radical prostatectomy for localized prostate cancer (cT1-T3a N0 or NX M0 or MX) using pre-operative factors, to assist the physician and patient in deciding whether or not radical prostatectomy is an acceptable treatment option. These nomograms can be used in clinical decision making by the clinician and patient and can be used to identify patients at high risk of disease recurrence who may benefit from neoadjuvant treatment protocols. Accordingly, one embodiment of the invention is directed to a method for predicting the probability of recurrence of prostate cancer following radical prostatectomy in a patient diagnosed as having prostate cancer. The method comprises correlating a selected set of pre-operative factors determined for each of a plurality of persons previously diagnosed with prostatic cancer and having been treated by radical prostatectomy with the incidence of recurrence of prostatic cancer for each person of the plurality of persons, so as to generate a functional representation of the correlation. The selected set of pre-operative factors includes, but is not limited to, pre-treatment blood TGF-β₁, IL6sR, sVCAM, VEGF, UPAR, UPA, and/or PSA; primary Gleason grade in the biopsy specimen; secondary Gleason grade in the biopsy specimen; Gleason sum; pre-radical prostatectomy therapy (e.g., hormone or radiation); and/or clinical stage; and matching an identical set of pre-operative factors determined from the patient diagnosed as having prostatic cancer to the functional representation so as to predict the probability of recurrence of prostatic cancer, organ confined disease, extracapsular extension, seminal vesical involvement, and lymph node status in the patient following radical prostatectomy. In an alternative embodiment, combined Gleason grade may be used instead of primary and secondary Gleason grades. The combined grade in the biopsy specimen (Bx Gleason Grade) includes the Gleason grade of the most predominant pattern of prostate cancer present in the biopsy specimen (the primary Gleason grade) plus the second most predominant pattern (secondary Gleason grade), if that pattern comprises at least 5% of the estimated area of the cancer or the histologic sections of the biopsy specimen. The terms “correlation,” “correlate” and “correlating” include a statistical association between factors and outcome, and may or may not be equivalent to a calculation of a statistical correlation coefficient.

In one embodiment, the correlating includes accessing a memory storing the selected set of factors. In another embodiment, the correlating includes generating the functional representation and displaying the functional representation on a display. In one embodiment, the displaying includes transmitting the functional representation from a source. In one embodiment, the correlating is executed by a processor or a virtual computer program. In another embodiment, the correlating includes determining the selected set of pre-operative factors. In one embodiment, determining includes accessing a memory storing the set of factors from the patient. In another embodiment, the method further comprises transmitting the quantitative probability of an outcome, e.g., prostate cancer or recurrence of prostatic cancer. In yet another embodiment, the method further comprises displaying the functional representation on a display. In yet another embodiment, the method further comprises inputting the identical set of factors for the patient within an input device. In another embodiment, the method further comprises storing any of the set of factors to a memory or to a database.

In one embodiment, the functional representation is a nomogram and the patient may be one who has not previously been diagnosed with prostate cancer, who has not previously been treated for prostate cancer or is a pre-surgical candidate. In one embodiment, the plurality of persons comprises persons with recently diagnosed prostate cancer but not having undergone treatment, or those with clinically localized prostate cancer not treated previously by radiotherapy, cryotherapy and/or hormone therapy, who have subsequently undergone radical prostatectomy. In one embodiment, the probability of recurrence of prostate cancer is a probability of remaining free of prostatic cancer five years following radical prostatectomy. Disease recurrence may be characterized as an increased serum PSA level, preferably greater than or equal to 0.4 ng/mL. Alternatively, disease recurrence may be characterized by positive biopsy, bone scan, or other imaging test or clinical parameter. Recurrence may alternatively be characterized as the need for or the application of further treatment for the cancer because of the high probability of subsequent recurrence of the cancer.

In one embodiment, the nomogram is generated with a Cox proportional hazards regression model (Cox, 1972, the disclosure of which is specifically incorporated by reference herein). This method predicts survival-type outcomes using multiple predictor variables. The Cox proportional hazards regression method estimates the probability of reaching a certain end point, such as disease recurrence, over time. In another embodiment, the nomogram may be generated with a neural network model (Rumelhart et al., 1986, the disclosure of which is specifically incorporated by reference herein). This is a non-linear, feed-forward system of layered neurons which backpropagate prediction errors. In another embodiment, the nomogram may be generated with a recursive partitioning model (Breiman et al., 1984, the disclosure of which is specifically incorporated by reference herein). In yet another embodiment, the nomogram is generated with support vector machine technology (Cristianni et al., 2000; Hastie, 2001). In a further embodiment, e.g., for hormone refractory patients, an accelerated failure time model may be employed (Harrell, 2001). Other models known to those skilled in the art may alternatively be used. In one embodiment, the invention includes the use of software that implements Cox regression models or support vector machines to predict prostate cancer, or prostate cancer recurrence, disease-specific survival, disease-free survival and/or overall survival.

In one embodiment, the nomogram may comprise an apparatus for predicting probability of disease recurrence in a patient with prostatic cancer following a radical prostatectomy. The apparatus comprises a correlation of pre-operative factors determined for each of a plurality of persons previously diagnosed with prostatic cancer and having been treated by radical prostatectomy with the incidence of recurrence of prostatic cancer for each person of the plurality of persons, the pre-operative factors include pre-treatment plasma TGF-β₁, IL6sR, IL-6, IGBPF-2, IGBPF-3, sVCAM, VEGF, PSA, UPAR, UPA, and/or BPSA; primary Gleason grade in the biopsy specimen; secondary Gleason grade in the biopsy specimen; and/or clinical stage; and a means for matching an identical set of pre-operative factors determined from the patient diagnosed as having prostatic cancer to the correlation to predict the probability of recurrence of prostatic cancer in the patient following radical prostatectomy.

Another embodiment of the invention is directed to a pre-operative nomogram which incorporates pre-treatment plasma TGF-β₁, IL6sR, IL-6, IGBPF-2, IGBPF-3, sVCAM, PSA, UPAR, UPA, VEGF, and/or BPSA; Gleason grade in the biopsy specimen; secondary Gleason grade in the biopsy specimen; and/or clinical stage; as well as one or more of the following additional factors: 1) total length of cancer in the biopsy cores; 2) number of positive cores; and 3) percent of tumor, in a 12 core biopsy set, as well as with other routinely determined clinical factors. For example, and not by way of limitation, if available pre-operatively, one or more of the factors p53, Ki-67, p27 or E-cadherin may be included (Stapleton et al., 1998; Yang et al., 1998).

With respect to the total length of cancer in the biopsy cores, it is customary during biopsy of the prostate to take multiple cores systematically representing each region of the prostate. With respect to the percent of cancerous tissue that percentage is calculated as the total number of millimeters of cancer in the cores divided by the total number of millimeters of tissue collected.

The present invention further comprises a method to predict a pre-operative prognosis in a patient comprising matching a patient-specific set of pre-operative factors such as pre-treatment plasma TGF-β₁, IL6sR, IL-6, IGBPF-2, IGBPF-3, sVCAM, PSA, VEGF, BPSA, UPA, UPAR, primary Gleason grade in the biopsy specimen, secondary Gleason grade in the biopsy specimen, and/or clinical stage, and determining the pre-operative prognosis of the patient.

The nomogram or functional representation may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The apparatus comprising a nomogram may further comprise a storage mechanism, wherein the storage mechanism stores the nomogram; an input device that inputs the identical set of factors determined from a patient into the apparatus; and a display mechanism, wherein the display mechanism displays the quantitative probability of recurrence of prostatic cancer. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of recurrence of prostatic cancer, e.g., the display is separated from the processor such that the display receives the quantitative probability of recurrence of prostatic cancer. The apparatus may further comprise a database, wherein the database stores the correlation of factors and is accessible by the processor. The apparatus may further comprise an input device that inputs the identical set of factors determined from the patient diagnosed as having prostatic cancer into the apparatus. The input device stores the identical set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the identical set of factors determined from the patient diagnosed as having prostatic cancer, preferably the transmission medium is coupled to the processor and the correlation of factors. The processor may be a multi-purpose or a dedicated processor. The processor includes an object oriented program having libraries, said libraries storing said correlation of factors.

In addition to assisting the patient and physician in selecting an appropriate course of therapy, nomograms may be useful in clinical trials to identify patients appropriate for a trial, to quantify the expected benefit relative to baseline risk, to verify the effectiveness of randomization, to reduce the sample size requirements, and to facilitate comparisons across studies.

The invention will be further described by the following non-limiting examples.

### EXAMPLE 1

TGF-β₁ Measurements

Serum and plasma samples may be collected on an ambulatory basis, e.g., at least 4 weeks after transrectal guided needle biopsy of the prostate, typically performed on the morning of the scheduled day of surgery after a typical pre-operative overnight fast. Blood may be collected into Vacutainer® CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500×g. The top layer corresponding to plasma may be decanted using sterile transfer pipettes and immediately frozen and stored at −80° C. in polypropylene cryopreservation vials (Nalgene, Nalge Nunc International, Rochester, N.Y.). Prior to assessment, an additional centrifugation step of the plasma at 10,000×g for 10 minutes at room temperature for complete platelet removal may be performed. For quantitative measurements of platelet-poor plasma and serum TGF-β₁ levels, a quantitative sandwich enzyme immunoassay (Quantikine® Human TGF-β₁ Elisa kit, R&D Systems, Minneapolis, Minn.) may be used, that is specific for TGF-β₁ and does not cross-react with TGF-β₂ or TGF-β₃. Recombinant TGF-β₁ may be used as standard. Every sample was run in duplicate, and the mean may be used for data analysis. Differences between the two measurements are minimal, as shown the intra-assay precision coefficient of variation of only 4.73±1.87%.

TGF-β₁ Collection Formats

TGF-β₁ levels may be assessed from three synchronously drawn blood specimens obtained from 10 of the 44 healthy screening patients. Plasma may be separated using Vacutainer® K₃ ethylenediaminetetraacetic acid (EDTA) 5 mL tubes containing 0.057 mL of 15% K₃ EDTA solution, and Vacutainer® CPT™ 8 mL tubes containing sodium citrate (Becton Dickinson Vacutainer Systems, Franklin Lakes, N.J.). Serum may be separated using Vacutainer® Brand SST Serum Separator™ tubes (Becton Dickinson Vacutainer Systems, Franklin Lakes, N.J.). Specimens may be centrifuged at room temperature for 20 minutes at 1500×g, and plasma or serum decanted and frozen at −80° C. until assessment. Prior to assay, an additional centrifugation step at 10,000×g for 10 minutes at room temperature may be performed. Analysis of variance may be used to determine whether the collection format significantly affects measured TGF-β₁ levels.

Impact of Collection Formats on TGF-β₁ Levels

Mean TGF-β₁ levels, measured in Vacutainer®CPT™ citrate plasma, Vacutainer®K₃ EDTA plasma, and Vacutainer®BrandSST™ serum from synchronously drawn blood specimens of 10 consecutive, healthy screening patients were 4.21±1.16 ng/mL, 8.34±2.94 ng/mL, and 23.89±5.35 ng/mL, respectively. TGF-β₁ levels measured in serum are 3-times higher than those in measured in citrate platelet-poor plasma and 6-times higher than those measured in EDTA platelet-poor plasma. Although analysis of variance showed TGF-β₁ inter-collection format differences to be statistically significant (P values<0.001), TGF-β₁ levels measured in specimens collected by all three sample formats are found to be highly correlated with each other (P values<0.001). However, levels of TGF-β₁ measured in specimens from the two platelet-poor plasma formats are the most highly correlated (CC=0.987). Platelet-poor plasma from Vacutainer®CPT™ sodium citrate tubes was used for TGF-β₁ measurements.

Final Pathological Stage and Progression as a Function of TGF-β₁ and Other Parameters

In both an univariate and a multivariate logistic regression analysis that included pre-operative TGF-β₁, pre-operative PSA, clinical stage, and biopsy Gleason score, plasma TGF-β₁ levels (P=0.006; Hazard ratio 0.616, 95% CI 0.436-0.869) and biopsy Gleason grade (P=0.006; Hazard ratio 3.671, 95% CI 1.461-9.219) were significant predictors of organ-confined disease (Table 3). Overall, only 14% of patients (17 of 120) had cancer progression with a median post-operative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with plasma TGF-β₁ levels above the median (4.9 ng/mL) had a significantly increased probability of PSA-progression (P=0.0105). On univariate Cox proportional hazards regression analysis, plasma TGF-β₁ was associated with the risk of PSA progression (P<0.001) along with biopsy Gleason score (P=0.005, Table 3). In a pre-operative multivariate model that included pre-operative TGF-β₁, pre-operative PSA, clinical stage, and biopsy Gleason score, plasma TGF-β₁ level and Gleason score (P<0.001) were both independent predictors of disease progression.

TABLE 3

| | Univariate | | | Multivariate | | |
|---|---|---|---|---|---|---|
| | Hazard | | | Hazard | | |
| Varible | ratio | P | 95% CI | ratio | P | 95% CI |
| Pre-operative PSA levels* | 5.772 | 0.067 | 0.887-37.547 | 2.408 | 0.363 | 0.362-16.016 |
| Pre-operative TGFβ-₁ | 2.246 | <0.001 | 1.637-3.083 | 2.268 | <0.001 | 1.629-3.158 |
| levels | | | | | | |
| Biopsy Gleason Score† | 4.167 | 0.005 | 1.541-11.273 | 3.582 | 0.021 | 1.212-10.585 |
| Clinical Stage‡ | 1.850 | 0.226 | 0.684-5.002 | 1.646 | 0.351 | 0.578-4.687 |

*Pre-operative PSA levels were logarithmically transformed.
†Biopsy Gleason Score was categorized as grade 2 to 6 versus grade 7 to 10.
‡Clinical stage was categorized as T1 versus T2.

IGF-I, IGFBP-2, and IGFBP-3 Measurements

Serum and plasma samples may be collected on an ambulatory basis, e.g., at least 4 weeks after transrectal guided needle biopsy of the prostate, typically performed on the morning of the scheduled day of surgery after a typical pre-operative overnight fast. Blood may be collected into Vacutainer® CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500×g. The top layer corresponding to plasma may be decanted using sterile transfer pipettes and immediately frozen and stored at −80° C. in polypropylene cryopreservation vials (Nalge Nunc, Rochester, N.Y.). For quantitative measurements of serum and plasma IGF-I and IGFBP-3 levels, the DSL-10-5600ACTIVE®IGF-I Elisa kit and the DSL-10-6600ACTIVE®IGFBP-3 Elisa kit may be used, respectively (DSL, Webster, Tex.). For quantitative measurements of serum and plasma IGFBP-2 levels, the DSL-7100 IGFBP-2 Radioimmunoassay kit (DSL) may be used. The mean of at least duplicate samples is used for data analysis. Differences between the two measurements were minimal, as shown the intra-assay precision coefficient of variation of only 4.73±1.87% for IGF-I, 6.95±3.86% for IGFBP-2, and 8.78±4.07 for IGFBP-3.

IGFBP-2 and IGFBP-3 Collection Formats

IGFBP-2 and IGFBP-3 levels may be assessed in three synchronously drawn blood specimens obtained from 10 of the 44 healthy screening patients. Plasma may be separated using Vacutainer® K₃ ethylenediaminetetraacetic acid (EDTA) 5 mL tubes containing 0.057 mL of 15% K₃ EDTA solution, and Vacutainer® CPT™ 8 mL tubes containing sodium citrate (Becton Dickinson Vacutainer Systems, Franklin Lakes, N.J.). Serum may be separated using Vacutainer® Brand SST Serum Separator™ tubes (Becton Dickinson Vacutainer Systems, Franklin Lakes, N.J.). Specimens may be centrifuged at room temperature for 20 minutes at 1500×g, and plasma or serum decanted and frozen at −80° C. until assessment. Analysis of variance may be used to determine whether the collection format significantly affected measured IGFBP-2 and IGFBP-3 levels.

Impact of Collection Formats on IGFBP-2 and IGFBP-3 Levels

Mean IGFBP-2 and IGFBP-3 levels, measured in Vacutainer®CPT™ citrate plasma, Vacutainer®K₃ EDTA plasma, and Vacutainer®BrandSST™ serum from synchronously drawn blood specimens of 10 consecutive, healthy screening patients are shown in Table 4. IGFBP-2 and IGFBP-3 levels measured in citrate plasma were 26% and 28%, respectively, lower than those measured in EDTA plasma, and 37% and 39%, respectively, lower than those measured in serum. Although analysis of variance showed IGFBP-2 and IGFBP-3 inter-collection format differences to be statistically significant (P values<0.001), IGFBP-2 and IGFBP-3 levels measured in specimens collected by all three sample formats were found to be highly correlated with each other (P values<0.001). Similarly to previous results on IGF-I (Shariat, 2000), while statistically significant differences were found in absolute IGFBP-2 and IGFBP-3 levels measured in different collection formats, all three collection formats were highly correlated with each other. Plasma from Vacutainer®CPT™ sodium citrate tubes was used for IGF-I, IGFBP-2, and IGFBP-3 measurements.

TABLE 4

| Collection FormatError! | IGF BP-2 (ng/mL) | | IGF BP-3 (ng/mL) | |
|---|---|---|---|---|
| Bookmark not defined. | Mean | SD* | Mean | SD* |
| Citrate plasma | 359.3 | 18.1 | 3273 | 256 |
| EDTA plasma | 487.9 | 28.4 | 4566 | 376 |
| Serum | 567.8 | 31.0 | 5401 | 430 |
| | | | | Corre- |
| | | | | lation |
| | P | Correlation | P | Coeffi- |
| Collection Formats | value† | Coefficient‡ | value† | cient‡ |
| EDTA plasma and citrate | <0.001 | 0.79 | <0.001 | 0.81 |
| plasma | | | | |
| EDTA plasma and serum | <0.001 | 0.70 | <0.001 | 0.72 |
| Citrate plasma and serum | <0.001 | 0.73 | <0.001 | 0.78 |

*SD = Standard Deviation.
†P-values (two-sided) were calculated based on analysis of variance in a randomized complete block design for the assessment of the difference in IGF BP-2 and IGF BP-3 levels between collection formats.
‡Spearman correlation coefficients were used to assess the relationship between different collection formats.

Clinical and Pathological Characteristics

All patients had clinically localized (T1 or T2) disease, and the mean pre-operative TGF-β₁ and PSA levels were 5.4±2.0 ng/mL (median 4.9, range 1.66 to 15.1) and 9.5±6.3 ng/mL (median 8.2, range 2.1 to 49.0), respectively. Nine (7.5%) patients had PSA levels less than 4 ng/mL; 75 (62.5%) had PSA levels greater than or equal to 4 ng/mL and less than 10 ng/mL; and 36 (30.0%) had PSA levels greater than or equal to 10 ng/mL. Clinical and pathological characteristics are listed in Table 5. On univariate analysis, pre-treatment IGFBP-2 levels correlated with pathological stage (P<0.001) and grade (P=0.025) and IGF BP-3 levels correlated with IGF-1 levels (P<0.001).

TABLE 5
Pre-Operative Characteristics

| | | Biopsy Gleason | |
|---|---|---|---|
| Clinical stage | Patients N (%) | score | Patients N (%) |
| cT1 a + b | 1 (0.8) | 2-4 | 3 (2.5) |
| cT1 c | 41 (34.2) | 5-6 | 77 (64.2) |
| cT2 a | 46 (38.3) | 7 | 35 (29.2) |
| cT2 b | 16 (13.3) | 8-10 | 5 (4.1) |
| cT2 c | 16 (13.3) | | |

Post-Operative Characteristics

| Pathological | Patients | Pathologic Gleason | Patients |
|---|---|---|---|
| features | N (%) | score* | N (%) |
| Organ Confined | 79 (65.8) | 2-4 | 0 (0) |
| ECE only | 33 (27.5) | 5-6 | 59 (50.0) |
| SVI+ | 8 (6.7) | 7 | 56 (47.5) |
| LN+ | 2 (1.7) | 8-10 | 3 (2.5) |
| SM+ | 16 (13.3) | | |

ECE = Extracapsular extension.
SVI+ = Seminal vesicle invasion.
LN+ = Lymph node positive.
SM+ = Positive surgical margins.
*Gleason tumor grade unavailable for two patients, who did not undergo a prostatectomy because of grossly positive pelvic lymph nodes at the time of surgery.

Final Pathological Stage and Progression as a Function of IGFBP-2 and IGFBP-3 and Other Parameters

In a multivariate logistic regression analysis, pre-operative plasma IGFBP-2 levels (P=0.001), pre-operative serum PSA levels (P=0.034), and biopsy Gleason grade (P=0.005) were significant predictors of organ-confined disease. Overall, only 14% of patients (17 of 120) had cancer progression with a median post-operative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with pre-operative plasma IGFBP-2 levels below the median (437.4 ng/mL) had a significantly increased probability of PSA-progression (P=0.0310). However, there was no significant difference in PSA-progression-free survival between patients stratified by the median level of IGFBP-3 (3239 ng/mL; P=0.0587). On univariate Cox proportional hazards regression analysis (Table 6), plasma IGFBP-2 was associated with the risk of PSA progression (P=0.015) along with biopsy Gleason score (P=0.005). In a pre-operative multivariate model that included pre-operative IGFBP-2, pre-operative PSA, clinical stage, and biopsy Gleason score, plasma IGFBP-2 level and biopsy Gleason score were both independent predictors of disease progression (P=0.049 and P=0.035, respectively). In alternative models where IGFBP-2 was replaced by IGF-I, IGFBP-3, or both, biopsy Gleason score was the sole independent predictor of PSA progression (P values≦0.09). However when IGFBP-3 level was adjusted for IGFBP-2 level, IGFBP-3 became an independent predictor of disease progression (P values≦0.040) and the association of IGFBP-2 with the risk of prostate progression strengthened (P values≦0.039). When all three, IGF-I, IGFBP-2, and IGFBP-3 were adjusted for each other, IGFBP-2, IGFBP-3, and biopsy Gleason score were independent predictors of disease progression (P=0.031, P=0.035, and P=0.036, respectively; Table 6).

TABLE 6

| | Univariate | | | Multivariate | | |
|---|---|---|---|---|---|---|
| | Haz- | | | Haz- | | |
| | ard | | | ard | | |
| Variable | ratio | P | 95% CI | ratio | P | 95% CI |
| Pre- | 0.997 | 0.490 | 0.990-1.005 | 1.003 | 0.454 | 0.995-1.012 |
| Operative | | | | | | |
| IGF-I | | | | | | |
| levels | | | | | | |
| Pre- | 0.993 | 0.015 | 0.988-0.999 | 0.994 | 0.031 | 0.988-0.999 |
| Operative | | | | | | |
| IGFBP-2 | | | | | | |
| levels | | | | | | |
| Pre- | 0.946 | 0.53 | 0.895-1.001 | 0.926 | 0.035 | 0.836-0.995 |
| Operative | | | | | | |
| IGFBP-3 | | | | | | |
| levels | | | | | | |
| Pre- | 5.772 | 0.067 | 0.887-37.547 | 3.671 | 0.124 | 0.699-19.270 |
| Operative | | | | | | |
| PSA | | | | | | |
| levels* | | | | | | |
| Biopsy | 4.167 | 0.005 | 1.541-11.273 | 3.055 | 0.036 | 1.079-8.654 |
| Gleason | | | | | | |
| Score† | | | | | | |
| Clinical | 1.850 | 0.226 | 0.684-5.002 | 1.769 | 0.293 | 0.611-5.122 |
| Stage‡ | | | | | | |

*Pre-operative PSA levels were logarithmically transformed.
†Biopsy Gleason Score was categorized as grade 2 to 6 versus grade 7 to 10.
‡Clinical stage was categorized as T1 versus T2.

IGFBP-2 and IGFBP-3 in Healthy and Metastatic Patients

Plasma IGF-I levels in 19 patients with prostate cancer metastatic to regional lymph nodes (median 156 ng/mL, range 100-281), in the 10 patients with prostate cancer metastatic to bones (153 ng/mL, range 29-360), in the cohort of 120 prostatectomy patients (median 151 ng/mL, range 42-451), and in the 44 healthy screening patients (median 171 ng/mL, range 62-346) were not significantly different from each other (P=0.413). However, plasma IGF BP-2 levels in the prostatectomy patients (median 437 ng/mL, range 209-871), in the patients with lymph node metastases (median 437 ng/mL, range 299-532), and in the patients with bone metastases (median 407 ng/mL, range 241-592) were significantly higher then those in the healthy subjects (median 340 ng/mL, range 237-495; P values<0.006). Plasma IGFBP-2 levels in patients with clinically localized prostate cancer, with lymph node metastases, or with bone metastases were not significantly different from each other (P values>0.413). Plasma IGFBP-3 levels in patients with lymph node metastases (median 2689 ng/mL, range 1613-3655) and bone metastases (median 2555 ng/mL, range 1549-3213) were significantly lower than those in the cohort of 120 prostatectomy patients (median 3217 ng/mL, range 1244-5452) and in healthy subjects (median 3344 ng/mL, range 1761-5020; P values<0.031). However, plasma IGFBP-3 levels in the prostatectomy patients were not significantly different than those in healthy subjects (P=0.575).

### EXAMPLE 2

A similar analysis was conducted for IL-6 and IL6sR (using R&D Systems Quantikine kits for IL-6 and IL6sR, catalog numbers DR6050 and DR600, respectively) and it was found that the pre-operative plasma levels of IL-6 and IL6sR were correlated with clinical and pathological parameters in the 120 patients who underwent radical prostatectomy (Tables 7-8). Plasma IL-6 and IL6sR levels in patients with bone metastases were significantly higher than those in healthy subjects, in prostatectomy patients, or in patients with lymph node metastases (P values≦0.001). In a pre-operative model that included IL-6 or IL6sR in addition to Partin nomogram variables, pre-operative plasma IL-6, IL6sR, and biopsy Gleason score were independent predictors of organ-confined disease (P values≦0.01) and PSA progression (P values≦0.028). In an alternative model that included both IL-6 and IL6sR, only pre-operative plasma IL6sR remained an independent predictor of PSA progression (P=0.038). Thus, IL-6 and IL6sR levels are elevated in men with prostate cancer metastatic to bone. In patients with clinically localized prostate cancer, the pre-operative plasma level of IL-6 and IL6sR are associated with markers of more aggressive prostate cancer and are predictors of biochemical progression after surgery.

TABLE 7
Pre-Operative Features

| | Univariate | | | Multivariate | | |
|---|---|---|---|---|---|---|
| | Hazard | | | Hazard | | |
| | ratio | P | 95% CI | ratio | P | 95% CI |
| Pre-Operative | 5.772 | 0.067 | 0.887-37.547 | 4.197 | 0.131 | 0.652-27.017 |
| PSA levels* | | | | | | |
| Pre-Operative IL-6 | 2.291 | <0.001 | 1.678-3.128 | 1.226 | <0.001 | 1.114-1.3498 |
| levels | | | | | | |
| Biopsy Gleason | 4.167 | 0.005 | 1.541-11.273 | 2.063 | 0.185 | 0.707-6.020 |
| Sum† | | | | | | |
| Clinical Stage‡ | 1.850 | 0.226 | 0.684-5.002 | 1.085 | 0.977 | 0.347-2.798 |

*Pre-operative PSA levels were logarithmically transformed.
†Biopsy Gleason sum was categorized as grade 2 to 6 versus grade 7 to 10.
‡Clinical stage was categorized as T1 versus T2.

TABLE 8
Pre-Operative Features

| | Univariate | | | Multivariate | | |
|---|---|---|---|---|---|---|
| | Hazard | | | Hazard | | |
| | ratio | P | 95% CI | ratio | P | 95% CI |
| Pre-Operative | 5.772 | 0.067 | 0.887-37.547 | 7.083 | 0.044 | 1.051-47.726 |
| PSA levels* | | | | | | |
| Pre-Operative IL-6 | 1.260 | <0.001 | 1.154-1.375 | 2.174 | <0.001 | 1.550-3.048 |
| levels | | | | | | |
| Biopsy Gleason | 4.167 | 0.005 | 1.541-11.273 | 3.218 | 0.026 | 1.148-9.025 |
| Sum† | | | | | | |
| Clinical Stage‡ | 1.850 | 0.226 | 0.684-5.002 | 1.135 | 0.814 | 0.396-3.254 |

*Pre-operative PSA levels were logarithmically transformed.
†Biopsy Gleason sum was categorized as grade 2 to 6 versus grade 7 to 10.
‡Clinical stage was categorized as T1 versus T2.

Association of Pre- and Post-Operative Plasma Levels of TGF-β₁, IL-6 and IL6sR with Clinical and Pathologic Characteristics

Clinical and pathologic characteristics of the 302 consecutive prostatectomy patients and association with pre- and post-operative plasma TGF-β₁, IL-6 and IL6sR levels are shown in Table 9.

TABLE 9

| | | TGF-β₁ (ng/mL) | | | | IL-6 (pg/mL) | | | | IL-6sR (ng/mL) | | | |
|---|---|---|---|---|---|---|---|---|---|---|---|---|---|
| | | Pre-operative | | Post-operative | | Pre-operative | | Post-operative | | Pre-operative | | Post-operative | |
| | No. Pts | Median | | Median | | Median | | Median | | Median | | Median | |
| | (%) | (Range) | P* | (Range) | P* | (Range) | P* | (Range) | P* | (Range) | P* | (Range) | P* |
| Prostatectomy | 302 | 3.9 | | 3.2 | | 1.9 | | 1.5 (0.0-7.3) | | 26.3 | | 20.6 | |
| patients | | (1.0-19.8) | | (0.5-18.1) | | (0.0-8.0) | | | | (10.4-48.2) | | (7.9-46.1) | |
| Clinical stage | | | | | | | | | | | | | |
| T1 | 141 (47) | 3.8 | .355 | 3.2 | .909 | 1.9 | .922 | 1.3 (0.0-7.7) | .171 | 24.7 | .190 | 19.7 | .135 |
| | | (1.0-19.3) | | (1.0-18.1) | | (0.0-7.6) | | | | (11.4-42.7) | | (7.9-45.0) | |
| T2 | 151 (50) | 3.9 | | 3.2 | | 1.9 | | 1.6 (0.0-6.3) | | 26.7 | | 20.9 | |
| | | (1.0-19.8) | | (0.5-13.9) | | (0.0-8.0) | | | | (10.4-48.2) | | (8.8-46.1) | |
| T3a | 10 (3) | 4.1 | | 3.4 | | 1.4 | | 1.4 (0.0-3.4) | | 24.8 | | 21.5 | |
| | | (2.8-17.0) | | (1.1-14.3) | | (0.4-4.4) | | | | (15.1-39.7) | | (10.5-28.4) | |
| Biopsy | | | | | | | | | | | | | |
| Gleason sum | | | | | | | | | | | | | |
| 2-6 | 199 (66) | 3.7 | .077 | 3.1 | .104 | 1.8 | .175 | 1.4 (0.0-7.7) | .251 | 25.3 | .087 | 20.1 | .075 |
| | | (1.0-19.8) | | (0.6-18.1) | | (0.0-8.0) | | | | (11.4-48.2) | | (7.9-46.1) | |
| 7-10 | 103 (34) | 4.2 | | 3.3 | | 2.0 | | 1.6 (0.0-5.6) | | 27.6 | | 21.6 | |
| | | (1.0-17.3) | | (0.5-14.3) | | (0.0-6.6) | | | | (10.4-45.9) | | (8.8-45.0) | |
| RP | | | | | | | | | | | | | |
| extraprostatic | | | | | | | | | | | | | |
| extension only† | | | | | | | | | | | | | |
| Negative | 195 (65) | 3.4 | .028 | 2.7 | <.001 | 1.8 | .066 | 1.5 (0.0-7.7) | .251 | 24.8 | .076 | 19.6 | .434 |
| | | (1.0-15.9) | | (0.5-18.1) | | (0.0-8.0) | | | | (10.4-45.9) | | (7.9-46.1) | |
| Positive | 105 (35) | 4.3 | | 3.8 | | 2.1 | | 1.5 (0.0-5.2) | | 27.0 | | 21.3 | |
| | | (1.3-19.8) | | (0.8-14.3) | | (0.0-6.6) | | | | (12.0-48.2) | | (8.8-45.0) | |
| RP seminal | | | | | | | | | | | | | |
| vesicle | | | | | | | | | | | | | |
| involvement† | | | | | | | | | | | | | |
| Negative | 279 (93) | 3.7 | .029 | 2.9 | .023 | 1.9 | .326 | 1.5 (0.0-7.7) | .434 | 25.5 | .698 | 21.6 | .427 |
| | | (1.0-19.8) | | (0.5-18.1) | | (0.0-8.0) | | | | (10.4-48.2) | | (7.9-46.1) | |
| Positive | 21 (7) | 4.6 | | 3.6 | | 2.0 | | 1.4 (0.9-3.6) | | 27.3 | | 19.5 | |
| | | (1.7-17.0) | | (1.2-14.3) | | (0.4-4.0) | | | | (11.7-41.6) | | (8.8-45.0) | |
| RP surgical | | | | | | | | | | | | | |
| margin† | | | | | | | | | | | | | |
| Negative | 260 (87) | 3.9 | .304 | 3.2 | .756 | 1.9 | .278 | 1.4 (0.0-6.3) | .987 | 26.0 | .782 | 21.6 | .202 |
| | | (1.0-19.8) | | (0.5-18.1) | | (0.0-8.0) | | | | (10.4-48.2) | | (7.9-46.1) | |
| Positive | 40 (13) | 3.8 | | 3.1 | | 2.0 | | 1.5 (0.0-7.7) | | 26.8 | | 18.4 | |
| | | (1.3-7.9) | | (0.8-5.2) | | (0.0-6.6) | | | | (11.7-43.8) | | (8.8-38.2) | |
| RP Gleason | | | | | | | | | | | | | |
| sum† | | | | | | | | | | | | | |
| 2-6 | 147 (49) | 3.8 | .912 | 3.0 | .117 | 1.7 | .014 | 1.4 (0.0-7.7) | .333 | 23.5 | .034 | 20.7 | .147 |
| | | (1.0-19.3) | | (0.6-18.1) | | (0.0-8.0) | | | | (11.4-45.4) | | (9.8-45.2) | |
| 7-10 | 153 (51) | 3.9 | | 3.4 | | 2.1 | | 1.6 (0.0-5.6) | | 28.6 | | 20.6 | |
| | | (1.0-19.8) | | (0.5-14.3) | | (0.0-6.6) | | | | (10.4-48.2) | | (7.9-46.1) | |
| RP lymph node | | | | | | | | | | | | | |
| metastases | | | | | | | | | | | | | |
| Negative | 296 (98) | 3.8 | <.001 | 3.0 | <.001 | 1.8 | .005 | 1.3 (0.0-7.7) | .084 | 24.4 | <.001 | 19.3 | .101 |
| | | (1.0-19.8) | | (0.5-18.1) | | (0.0-8.0) | | | | (10.4-37.8) | | (7.8-46.1) | |
| Positive | 6 (2) | 7.1 | | 6.5 | | 2.6 | | 1.6 (0.9-5.6) | | 29.8 | | 21.0 | |
| | | (3.3-17.3) | | (3.3-14.3) | | (1.4-7.6) | | | | (17.0-44.3) | | (10.5-39.9) | |
| RP DNA | | | | | | | | | | | | | |
| ploidy‡ | | | | | | | | | | | | | |
| Diploid | 125 (49) | 3.6 | .151 | 3.0 | .543 | 1.9 | .807 | 1.4 (0.0-5.2) | .288 | 26.0 | .804 | 20.8 | .643 |
| | | (1.1-15.9) | | (0.8-18.1) | | (0.0-6.5) | | | | (10.4-44.3) | | (11.4-46.1) | |
| Aneuploid or | 129 (51) | 4.0 | | 3.3 | | 1.9 | | 1.6 (0.0-4.2) | | 26.6 | | 19.5 | |
| tetraploid | | (1.0-19.8) | | (1.1-14.3) | | (0.0-8.0) | | | | (12.1-43.8) | | (7.9-36.1) | |

| TGF-β₁ | | IL-6 | | IL-6 sR | |
|---|---|---|---|---|---|
| Pre-operative | Post-operative | Pre-operative | Post-operative | Pre-operative | Post-Operative |

| | CC§ | P | CC§ | P | CC§ | P | CC§ | P | CC§ | P | CC§ | P |
|---|---|---|---|---|---|---|---|---|---|---|---|---|
| Age | 0.024 | .616 | 0.025 | .679 | 0.042 | .379 | 0.080 | .239 | 0.022 | .650 | 0.091 | .181 |
| Pre-operative PSA | .469 | .004 | 0.055 | .358 | 0.177 | <.001 | 0.077 | .254 | 0.201 | .011 | 0.057 | .401 |
| RP tumor volume \|\| | 0.109 | .095 | 0.112 | .159 | 0.172 | .018 | 0.068 | .454 | 0.198 | .016 | 0.046 | .610 |
| Pre-operative TGF-β₁ | — | — | 0.451 | <.001 | 0.116 | .019 | 0.091 | .069 | 0.193 | .038 | 0.088 | .207 |
| Post-operative TGF-β₁ | 0.451 | <.001 | — | — | 0.107 | .079 | 0.126 | .075 | 0.077 | .206 | 0.002 | .981 |
| Pre-operative IL-6 | 0.116 | .019 | 0.107 | .079 | — | — | 0.514 | <.001 | 0.443 | <.001 | .209 | .002 |
| Post-operative IL-6 | 0.091 | .069 | 0.126 | .075 | 0.514 | <.001 | — | — | 0.188 | .006 | 0.203 | .003 |
| Pre-operative IL-6sR | 0.193 | .038 | 0.077 | .206 | 0.443 | <.001 | 0.188 | .006 | — | — | 0.756 | <.001 |
| Post-operative IL-6sR | 0.088 | .207 | 0.002 | .981 | 0.209 | .002 | 0.203 | .003 | 0.756 | <.001 | — | — |

RP = Radical prostatectomy.
CC = Correlation coefficient
*Mann Whitney U test.
†RP extracapsular extension status, RP seminal vesicle involvement status, RP surgical margin status, and RP Gleason sum were not available for 2 patients, who did not undergo a prostatectomy because of positive pelvic lymph nodes at the time of surgery.
‡DNA ploidy was unavailable for 48 patients.
§Spearman's correlation coefficients.
|| Radical prostatectomy tumor volume was unavailable for 47 patients.

Pre-operative and post-operative plasma TGF-β₁ levels were elevated in patients with extraprostatic extension (P=0.028 and P<0.001, respectively), seminal vesicle involvement (P=0.029 and P=0.023, respectively), and regional lymph node metastases (P<0.001 and P<0.001, respectively). Preoperative IL-6 and IL6sR levels were elevated in patients with prostatectomy Gleason sum ≧7 (P=0.014 and P=0.034, respectively) and regional lymph node metastases (P=0.005 and P<0.001, respectively). The mean pre-operative PSA was 8.9±7.0 ng/mL (median 7.1, range 0.2 to 59.9). Pre-treatment TGF-β₁, IL-6, and IL6sR levels were positively correlated with pre-operative PSA levels (P=0.004, P<0.001, and P=0.011, respectively). Pre-treatment IL-6 and IL6sR levels were also positively correlated with prostatic tumor volume (P=0.018 and P=0.016, respectively). Post-operative IL-6 and IL6sR levels were not associated with any of the clinical or pathologic parameters.

In univariable logistic regression analyses, pre-operative TGF-β₁ levels predicted organ confined disease (P=0.017, Hazard ratio 0.902, 95% CI 0.828-0.982), but pre-operative IL-6 and IL6sR did not (P=0.118 and P=0.079, respectively). In a pre-operative multivariable model, clinical stage (P=0.035) and biopsy Gleason sum (P<0.001) were the only predictors of organ confined disease, when adjusted for the effects of pre-operative PSA (P=0.087), pre-operative TGF-β₁ (P=0.112), pre-operative IL-6 (P=0.639), and pre-operative IL6sR (P=0.725).

Association of Pre- and Post-Operative Plasma Levels of TGF-β₁, IL-6 and IL6sR with Prostate Cancer Progression

Overall, only 14% of patients (43 of 302) had cancer progression with a median post-operative follow-up of 50.7 months (range 1.2 to 73.5). The overall PSA progression-free survival was 88.8±1.5% (Standard error, SE) at 3 years and 85.1±1.9% (SE) at 5 years. On univariable Cox proportional hazards regression analyses, pre- and post-operative TGF-β₁ (P<0.001), pre-operative IL-6 (P<0.001), pre-operative IL6sR (P<0.001), pre-operative PSA (P<0.001), biopsy and prostatectomy Gleason sum (P<0.001 and P<0.001, respectively), extraprostatic extension (P<0.001), seminal vesicle involvement (P<0.001), and surgical margin status (P<0.001) were associated with cancer progression, but post-operative IL-6 (P=0.162), post-operative IL6sR (P=0.079), and clinical stage (P=0.103) were not.

TABLE 11

| | Model 1 | | | Model 2 | | | Model 3 | | |
|---|---|---|---|---|---|---|---|---|---|
| | Hazard ratio | 95% CI | P | Hazard ratio | 95% CI | P | Hazard ratio | 95% CI | P |
| Pre-Operative PSA* | 1.323 | 0.872-2.009 | .183 | 1.291 | 1.128-2.446 | .174 | 1.577 | 0.977-2.546 | .062 |
| Extraprostatic extension | 1.085 | 0.581-2.027 | .798 | 0.974 | 0.487-1.948 | .941 | 1.046 | 0.432-1.765 | .706 |
| Seminal vesicle involvement | 2.212 | 1.138-4.699 | .020 | 1.202 | 0.562-2.571 | .235 | 1.269 | 0.572-2.816 | .258 |
| RP Gleason sum† | 4.281 | 1.838-9.975 | <.001 | 4.042 | 1.657-9.855 | <.001 | 3.706 | 1.494-9.191 | .005 |
| Surgical margin status | 2.595 | 1.232-4.276 | .009 | 1.453 | 0.772-2.734 | .107 | 1.501 | 0.784-2.874 | .114 |
| Pre-Operative IL-6 | 1.629 | 0.989-1.495 | .055 | — | — | — | 1.122 | 0.953-1.081 | .332 |
| Pre-Operative IL-6sR | 1.843 | 1.001-1.088 | .045 | — | — | — | 1.215 | 0.953-1.452 | .268 |
| Pre-Operative TGF-β₁ | 1.151 | 1.057-2.253 | <.001 | — | — | — | 1.058 | 0.870-1.285 | .574 |
| Post-Operative IL-6 | — | — | — | 1.154 | 0.923-1.443 | .208 | 1.031 | 0.790-1.346 | .822 |
| Post-Operative IL-6sR | — | — | — | 0.992 | 0.952-1.034 | .698 | 0.984 | 0.932-1.039 | .566 |
| Post-Operative TGF-β₁ | — | — | — | 2.305 | 1.188-3.532 | <.001 | 2.241 | 1.247-3.356 | .013 |

RP = radical prostatectomy
*Pre-operative PSA level had a skewed distribution and therefore was modeled with a log transformation.
†Radical prostatectomy Gleason sum was evaluated as grade 2 to 6 versus grade 7 to 10.

In a pre-operative multivariable model, pre-operative TGF-β₁ (P=0.010, Hazard ratio 1.710, 95% CI 1.078-2.470), IL6sR (P=0.038, Hazard ratio 1.515, 95% CI 1.011-2.061), and biopsy Gleason sum (P<0.001, Hazard ratio 2.896, 95% CI 1.630-5.145) were associated with cancer progression when adjusted for the effects of pre-operative PSA (P=0.058), pre-operative IL-6 (P=0.062), and clinical stage (P=0.837).

Pre- and post-operative TGF-β₁, IL-6 and IL6sR were analyzed in separate post-operative multivariable Cox proportional hazards regression analyses that also included extracapsular extension, seminal vesicle involvement, surgical margin status, pathologic Gleason sum, and pre-operative PSA. In the first model that included pre-operative levels of the candidate markers, pre-operative TGF-β₁ (P<0.001) and IL6sR (P=0.045) along with prostatectomy Gleason sum (P<0.001), seminal vesicle involvement (P=0.020), and surgical margin status (P=0.009) were associated with cancer progression. In the second model that included post-operative levels of the candidate markers, only post-operative TGF-β₁ (P<0.001) and prostatectomy Gleason sum (P<0.001) were associated with disease progression. In the third model that included pre- and post-operative levels of TGF-β₁, IL-6 and IL6sR, only post-operative TGF-β₁ (P=0.013) and prostatectomy Gleason sum (P=0.005) were associated with prostate cancer progression.

TABLE 12

| | | TGF-β₁ (ng/mL) | | | | IL-6 (pg/mL) | | | | IL-6sR (ng/mL) | | | |
|---|---|---|---|---|---|---|---|---|---|---|---|---|---|
| | | | | Percent | | | | Percent | | | | Percent | |
| | No. | Pre- | Post- | De- | | Pre- | Post- | De- | | Pre- | Post- | De- | |
| | Pts. | Operative | Operative | crease | P* | Operative | operative | crease | P* | Operative | Operative | crease | P* |
| All patients | 302 | 3.9 | 3.2 | 18% | .029 | 1.9 (0.0-8.0) | 1.5 (0.0-7.3) | 21% | <.001 | 26.3 | 20.6 | 22% | <.001 |
| | | (1.0-19.8) | (0.5-18.1) | | | | | | | (10.4-48.2) | (7.9-46.1) | | |
| Patients who | 43 | 4.7 | 4.3 | 9% | .074 | 2.3 (1.0-8.0) | 1.6 (0.0-7.3) | 30% | <.001 | 30.6 | 22.3 | 27% | <.001 |
| experienced | | (1.6-19.8) | (1.2-18.1) | | | | | | | (13.2-48.2) | (7.9-46.1) | | |
| cancer | | | | | | | | | | | | | |
| progression | | | | | | | | | | | | | |
| Patients who | 259 | 3.6 | 2.4 | 33% | <.001 | 1.7 (0.0-7.1) | 1.4 (0.0-5.8) | 18% | .042 | 24.1 | 20.1 | 17% | .034 |
| did not | | (1.0-10.3) | (0.5-8.3) | | | | | | | (10.4-32.3) | (7.9-33.4) | | |
| experience | | | | | | | | | | | | | |
| cancer | | | | | | | | | | | | | |
| progression | | | | | | | | | | | | | |

*Wilcoxon signed-rank test.

Pre-Versus Post-Prostatectomy TGF-β₁, IL-6 and IL6sR Levels

Overall, post-operative TGF-β₁, IL-6, and IL6sR levels were all lower than pre-operative levels (P=0.029, P=<0.001, and P<0.001, respectively; Table 12). In the subgroup of patients who experienced disease progression, post-operative IL-6 and IL6sR levels were both lower than pre-operative IL-6 and IL6sR levels (P<0.001 and P<0.001, respectively). However, post-operative TGF-β₁ levels were not different than pre-operative TGF-β₁ levels (P=0.074). In the subgroup of patients who did not experience cancer progression, pre-operative levels of TGF-β₁, IL-6, and IL6sR declined after surgery P<0.001, P=0.042, and P=0.034, respectively).

### EXAMPLE 3

VEGF and sVCAM-1 Measurements

Plasma samples may be collected after a pre-operative overnight fast, e.g., on the morning of the day of surgery, at least 4 weeks after transrectal guided needle biopsy of the prostate. Blood may be collected into Vacutainer®CPT™ 8 mL tubes containing 0.1 mL of Molar sodium citrate (Becton Dickinson Vacutainer Systems, Franklin Lakes, N.J.) and centrifuged at room temperature for 20 minutes at 1500×g. The top layer corresponding to plasma may be decanted using sterile transfer pipettes. The plasma is immediately frozen and stored at −80° C. in polypropylene cryopreservation vials (Nalgene, Nalge Nunc, Rochester, N.Y.). It has been previously found that VEGF levels are higher when measured in serum than when measured in plasma. Since VEGF is present in platelet granules and is released upon platelet activation, the higher levels of VEGF in serum are likely due at least in part to release from damaged platelets, making the quantification of non-platelet derived VEGF less accurate (Spence et al., 2002). Therefore, for VEGF, prior to assessment, an additional centrifugation step of the plasma may be performed at 10,000×g for 10 minutes at room temperature for complete platelet removal (Adams et al., 2000). For quantitative measurements of VEGF and sVCAM-1 levels, quantitative immunoassays may be employed (R&D Systems, Minneapolis, Minn.). Every sample may be run at least in duplicate, and the mean of the results may be used. Differences between the two measurements for both VEGF and sVCAM-1 were minimal (intra-assay precision coefficients of variation: 8.49±11.10% and 4.86±6.31%, respectively).

Plasma VEGF and sVCAM-1 in Patients with Prostate Cancer Metastases

Plasma VEGF and sVCAM-1 levels were assessed in nine patients with bone scan-proven, metastatic prostate cancer, and 215 patients diagnosed with clinically localized prostate cancer. Neither of these patients were treated with either hormonal or radiation therapy before plasma collection. Plasma VEGF and sVCAM-1 levels in patients with prostate cancer metastatic to bones (median 31.3, range 15.3-227.1 and median 648.7, range 524.8-1907.1, respectively) were higher than those in patients with clinically localized disease (median 9.9, range 2.0-166.9 and median 581.8, range 99.0-2068.3, respectively; P values<0.001). Plasma levels for healthy controls were within the normal range reported by the ELISA company for both VEGF and sVCAM-1 (median 2.24, range 1.6 to 3.0 and median 555.0, range 398.0 to 712.0, P values<0.001 respectively)

Association of Pre-Operative Plasma VEGF and sVCAM-1 with Clinical and Pathologic Characteristics of Prostate Cancer

Clinical and pathologic characteristics of 215 prostatectomy patients and association with pre-operative plasma VEGF and sVCAM-1 levels are shown in Table 13. Pre-operative VEGF and sVCAM-1 levels were both elevated in patients with lymph node involvement (P<0.001 and P=0.012, respectively). However only pre-operative plasma VEGF was elevated in patients with biopsy and final Gleason sum ≧7 (P=0.036 and P=0.040, respectively) and extraprostatic extension (P=0.047). The mean pre-operative PSA was 9.15±1.01 ng/mL (median 7.3, range 1.1 to 60.1). Sixty-two patients (28%) had PSA levels of 10 ng/mL and beyond. On univariate logistic regression analyses pre-operative plasma VEGF levels were associated with organ-confined disease (Hazard ratio 0.991, 95% CI 0.983-0.998, P=0.016) and lymph node involvement (Hazard ratio 1.033, 95% CI 1.019-1.047, P<0.001), whereas pre-operative plasma sVCAM-1 levels were not (P=0.367 and P=0.063, respectively). On multivariate logistic regression analyses (Table 4), pre-operative plasma VEGF was associated with prostate cancer involvement of the lymph nodes (P<0.001) but not with confinement of the cancer to the prostate (P=0.528), when adjusted for the effects of standard pre-operative features and pre-operative plasma sVCAM-1.

TABLE 13

| | | Pre-operative | | | Pre-operative | | |
|---|---|---|---|---|---|---|---|
| | | VEGF (pg/mL) | | | sVCAM-1 (ng/mL) | | |
| | No. Pts (%) | Median | Range | P | Median | Range | P |
| Healthy Controls | 40 | 2.2 | 1.6-3.0 | | 555.0 | 328.0-712.0 | |
| Prostatectomy patients | 215 | 9.9 | 2.0-166.9 | <.001 | 581.8 | 116.0-2068.3 | .290 |
| Clinical stage | | | | | | | |
| T1c | 97(45) | 9.3 | 4.1-166.9 | | 493.8 | 116.0-2068.3 | |
| T2a | 56(26) | 9.6 | 4.1-153.4 | | 481.7 | 178.0-1807.6 | |
| T2b | 36(17) | 12.2 | 2.0-151.8 | | 542.8 | 203.3-1144.9 | |
| T2c | 23(11) | 14.1 | 4.5-97.4 | | 403.7 | 99.4-1201.1 | |
| T3a | 3(1) | 34.1 | 9.9-134.4 | .054 | 345.40 | 314.3-888.7 | .203 |
| Biopsy Gleason sum | | | | | | | |
| 2-6 | 143(67) | 9.6 | 2.0-166.9 | | 477.80 | 402.1-1807.6 | |
| 7-10 | 72(33) | 13.2 | 4.8-153.4 | .036 | 531.05 | 116.0-2068.3 | .311 |
| RP extraprostatic | | | | | | | |
| extension only‡ | | | | | | | |
| Negative | 139(65) | 9.6 | 2.0-166.9 | | 475.90 | 402.1-1807.6 | |
| Positive | 74(35) | 12.4 | 4.4-151.8 | .047 | 524.20 | 99.4-2068.3 | .234 |
| RP seminal vesicle | | | | | | | |
| involvement‡ | | | | | | | |
| Negative | 198(93) | 9.9 | 2.0-166.9 | | 490.90 | 402.1-2068.3 | |
| Positive | 15(7) | 12.1 | 4.4-134.32 | .438 | 501.40 | 214.4-888.7 | .842 |
| RP surgical margin‡ | | | | | | | |
| Negative | 180(85) | 9.6 | 2.0-166.9 | | 482.60 | 402.1-1807.6 | |
| Positive | 33(15) | 12.1 | 4.8-125.1 | .116 | 515.00 | 99.4-2068.3 | .501 |
| RP Gleason sum‡ | | | | | | | |
| 2-6 | 91(43) | 9.3 | 2.0-159.5 | | 501.06 | 99.4-1807.6 | |
| 7-10 | 122(57) | 10.94 | 4.4-166.9 | .040 | 499.20 | 402.1-2068.3 | .843 |
| RP regional lymph | | | | | | | |
| node metastases | | | | | | | |
| Negative | 204(95) | 9.6 | 4.0-2068.3 | | 476.90 | 402.1-2068.3 | |
| Positive | 11(5) | 29.8 | 20.2-153.4 | <.001 | 611.50 | 490.2-1439.2 | .012 |

| | CC§ | P | CC§ | P |
|---|---|---|---|---|
| Age | 0.133 | .051 | 0.149 | .090 |
| Pre-operative PSA | 0.119 | .081 | −0.025 | .717 |
| Pre-operative VEGF | — | — | −0.005 | .940 |
| Pre-operative sVCAM-1 | −0.005 | .940 | — | — |
| RP tumor volume□ | 0.113 | .126 | 0.008 | .927 |

RP = Radical prostatectomy
CC = Correlation coefficient
‡RP extracapsular extension status, RP seminal vesicle involvement status, RP surgical margin status, and RP Gleason sum were not available for two patients, who did not undergo a prostatectomy because of positive pelvic lymph nodes at the time of surgery.
§Spearman's correlation coefficients.
□Radical prostatectomy tumor volume was unavailable for 61 prostatectomy patients

TABLE 14

| | Organ Confined Disease | | | Metastases to Regional Lymph Nodes | | |
|---|---|---|---|---|---|---|
| | Hazard Ratio | 95% CI | P | Hazard Ratio | 95% CI | P |
| Pre-operative VEGF | 0.997 | 0.988-1.006 | .528 | 1.036 | 1.018-1.053 | <.001 |
| Pre-operative sVCAM-1 | 1.000 | 0.999-1.001 | .455 | 1.002 | 0.999-1.004 | .090 |
| Pre-operative PSA* | 0.928 | 0.878-0.980 | .008 | 0.971 | 0.871-1.082 | .592 |
| Biopsy Gleason Sum† | 0.293 | 0.168-0.510 | <.001 | 2.603 | 0.553-12.247 | .226 |
| Clinical Stage | 0.771 | 0.580-1.025 | .073 | 2.584 | 1.167-5.720 | .019 |

*Pre-operative PSA level had a skewed distribution and therefore was modeled with a log transformation.
†Biopsy Gleason Sum was categorized as grade 2 to 6 versus grade 7 to 10.

Association of Pre-Operative Plasma VEGF and sVCAM-1 with Biochemical Progression after Radical Prostatectomy

Overall, 20% of patients (42 of 215) had cancer progression with a median post-operative follow-up of 60.1 months (range 2.5 to 86.3). The overall PSA progression-free survival was 86.0±2.4% (Standard error, SE) at 3 years, 79.3±3.0% (SE) at 5 years, and 76.9±3.3% (SE) at 7 years. On univariate and multivariate Cox proportional hazards regression analysis (Table 15), higher pre-operative plasma VEGF (P=0.005 and P=0.043, respectively) as well as biopsy Gleason sum ≧7 (P=0.001 and P=0.015, respectively) and pre-operative serum PSA (P<0.001 and P<0.001, respectively) were associated with the risk of PSA progression, when adjusted for the effects of clinical stage and pre-operative plasma sVCAM-1.

TABLE 15

| | Univariable | | | Multivariable | | |
|---|---|---|---|---|---|---|
| | Hazard | | | Hazard | | |
| | Ratio | 95% CI | P | Ratio | 95% CI | P |
| Pre- | 1.009 | 1.003-1.016 | .005 | 1.008 | 1.000-1.015 | .043 |
| operative | | | | | | |
| VEGF | | | | | | |
| Pre- | 1.001 | 0.999-1.001 | .122 | 1.001 | 0.999-1.002 | .066 |
| operative | | | | | | |
| sVCAM-1 | | | | | | |
| Pre- | 1.067 | 1.043-1.092 | <.001 | 1.058 | 1.032-1.085 | <.001 |
| operative | | | | | | |
| PSA* | | | | | | |
| Biopsy | 2.891 | 1.572-5.315 | .001 | 2.223 | 1.168-4.229 | .015 |
| Gleason | | | | | | |
| Sum† | | | | | | |
| Clinical | 0.915 | 0.684-1.224 | .548 | 0.879 | 0.651-1.188 | .402 |
| Stage | | | | | | |

*Pre-operative PSA level had a skewed distribution and therefore was modeled with a log transformation.
†Biopsy Gleason Sum was categorized as grade 2 to 6 versus grade 7 to 10.

### EXAMPLE 4

Several studies have conclusively shown that standard sextant biopsy (S6C) detects fewer prostate cancers compared to biopsy templates that include additional, laterally-directed biopsy cores (Gore et al., 2001; Chang et al., 1998). For example, Gore et al. (2001) demonstrated that sextant biopsies detected only 69% of the cancers identified by a systematic 12-core biopsy (S12C) regimen that included 6 additional, laterally directed cores, one each at the base, mid-portion, and apex of the prostate in addition to standard S6C. Since S6C fails to detect approximately one-third of cancers present, it seems inevitable that S6C would also perform poorly in predicting pathologic features of the prostate following radical prostatectomy; in fact, many studies have confirmed the poor performance of S6C in predicting post-prostatectomy pathology. These studies have assessed the predictive value of various biopsy parameters, including biopsy GS, number of positive cores, percent of tumor in the biopsy specimen, and total length of cancer in S6C set in predicting pathologic features of the prostatectomy specimen. Sebo et al. (2000) reported that percent of cores positive for cancer and biopsy Gleason score of sextant biopsy were independent, significant predictors of tumor volume. However, in that study the correlation coefficients were 27% and 11.6% (R² multiplied by 100), respectively. In another study, although cancer volume significantly correlated with the number of positive biopsies, percent of positive biopsies, total cancer length in the biopsy specimen, and Gleason grade 4/5, all correlation coefficients were less than 10% (Noguchi et al., 2001).

Despite these significant associations between S6C biopsy parameters and prostatectomy pathology, reliable algorithms that include S6C biopsy parameters to predict extracapsular extension (ECE) (Egawa et al., 1998), tumor volume (Noguchi et al., 2001), and pathologic Gleason score (pGS) (Narain et al., 2001) have not emerged. Noguchi et al. (2001) reported that there was a weak and disappointing correlation among all pathological features of 6 systematic biopsies and radical prostatectomy specimens. Cupp et al. (1995) also demonstrated the poor performance of S6C biopsies in predicting pathologic parameters of the radical prostatectomy specimen.

Material and Methods

Patient Population

All 228 patients who underwent a S12C biopsy at a single institution (Scott Department of Urology, Baylor College of Medicine, Houston, Tex.) and a subsequent radical retropubic prostatectomy by a member of the full-faculty were potential candidates for this analysis. S12C biopsy became the standard initial biopsy technique for the Baylor Prostate Center faculty. Two men initially treated with definitive radiotherapy and forty-eight who had a history of a prostate biopsy prior to their S12C biopsy were excluded. This left one hundred seventy-eight (178) men for analysis.

Prostate Needle Biopsy Technique

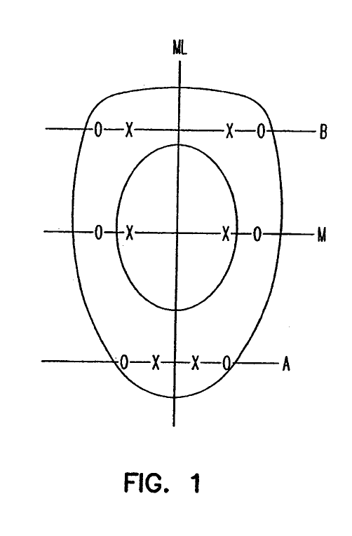

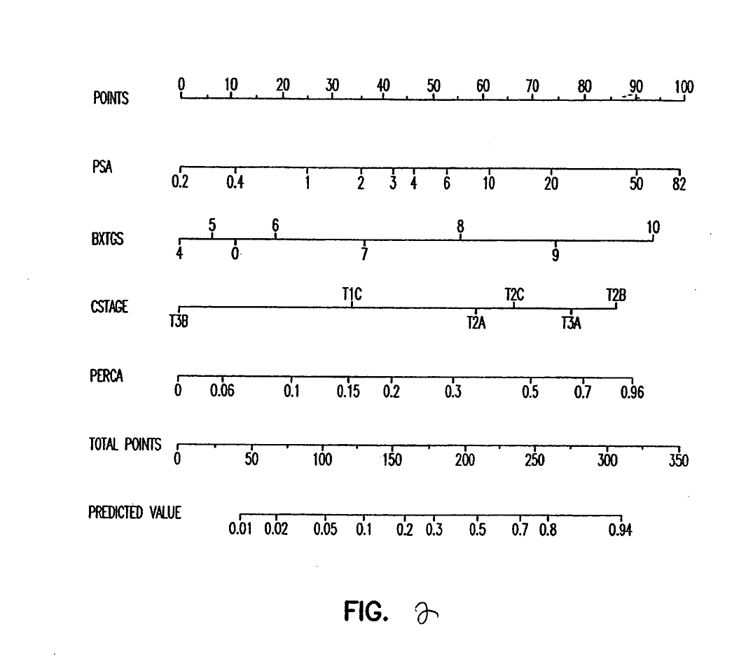

The S12C needle biopsy was performed as previously described (Gore et al., 2001). Briefly, a standard sextant biopsy as described by Hodge et al. (1989) was performed with the addition of laterally directed biopsies in the peripheral zone at the base, mid, and apex of the prostate (FIG. 1). Each biopsy core was individually identified as to its location of origin (base, mid, or apex; right or left; sextant or laterally-directed) using a 4-specimen cup technique and the use of red, green, and blue ink. Additional ultrasound, finger, or transitional zone directed biopsy cores performed at the discretion of the staff urologist were excluded from this study. All biopsies were performed in a standardized fashion by a staff urologist along with one of two ultrasound technicians, who served to help standardize the biopsy template across all patients. Gray scale transrectal ultrasonography was performed using the Hitachi (Hitachi Medical Systems, Tokyo, Japan) EUB-V33W 6.5 MHz end-fire probe. Biopsy cores were obtained using an 18 gauge needle with the ProMag (Manan Medical Systems, Northbrook, Ill.) 2.2 spring loaded gun. The entire prostate gland and transitional zone were measured in three dimensions, and the volume estimated using the prolate ellipsoid formula.

Pathology Specimens

In each biopsy specimen, the following variables were assessed and documented by a full-time faculty pathologist: total millimeter (mm) of cancer involvement of each core, total mm length of each core, and GS of the tumor identified in any core with tumor. Radical retropubic prostatectomies were performed at one of two teaching hospitals, either St. Luke's Episcopal Hospital (n=42), Houston, Tex., or The Methodist Hospital (n=136), Houston, Tex. Prostatectomy specimens at The Methodist Hospital were fixed and processed in the whole-mount technique with 5-mm transverse sections as previously described in Wheeler and Lebowitz (1994). Prostatectomy specimens at St. Luke's Hospital were serially sectioned into multiple levels and then subdivided into two or four pieces and submitted in entirety. pGS was assigned after review of the cross-sections. ECE was scored as a binary, categorical variable (with L3E and L3F considered positive, see Wheeler et al., 1998) after the extent of each cancer focus was identified. Total tumor volume (TTV) was calculated using a computerized planimetric method with Optimas software using the Bioscan image analysis system on all whole mount step sectioned prostatectomy specimens.

Prognostic Variables and Statistics

The comparison biopsy set groups included the sextant (FIG. 1, S6C X), the laterally directed systematic six cores (FIG. 1, L6C=O), and entire S12C biopsy set (FIG. 1, S12C=X+O). The percent of tumor involvement per biopsy set was derived using the formula: ((total percent of tumor in core 1)+(total percent of tumor in core 2)+(total percent of tumor in core 3)+ . . . /(total number of cores in the set))×100. The total cancer length of a biopsy set was the sum of all mm of cancer in that particular biopsy set. Biopsy GS was determined as the sum of the maximum primary and secondary Gleason grades for the biopsy set. Biopsy GS, number of positive cores, total length of cancer, and percent of tumor in each biopsy set group were examined for their ability to predict ECE, TTV, and pGS with Spearman's rho correlation coefficients.

Stepwise multiple regression analyses were performed to determine independent predictors of the prostatectomy pathology. Biopsy parameters from both the L6C and S6C sets were included this analysis. S12C set biopsy predictors were not included in this analysis because these parameters are not independent of the S6C and 6LC parameters, but simply mathematical manipulations of them. For instance, the S12C number of positive cores and total cancer length are the addition of the L6C and S6C parameters, the percent of tumor involvement is the addition of L6C and S6C percent tumor involvement divided by two, and the S12C biopsy GS is the sum of the maximum primary and secondary grades contained in the L6C and S6C sets. Statistical significance in this study was set as P<0.05. All reported P values are two-sided. All analyses were performed with the SPSS statistical package (SPSS version 10.0 for Windows).

The independent biopsy predictors of ECE, pGS, and TTV were utilized to construct a test to evaluate the sensitivity, specificity, and positive and negative predictive values for the presence of insignificant cancer as defined by described by Epstein et al. (1998). Specifically, insignificant tumors were defined as having a tumor volume of <0.5 cm³, confined to the prostate, and having a pGS less than 7. To minimize bias, the median results of the biopsy predictor variables were used as the cut-point values.

Results

The median age for the study cohort was 62 years, and the median total and % free PSA were 5.8 ng/ml and 24.7, respectively. The median TTV was 0.56 cc. 24.7% of the patients had ECE (Table 16).

S12C set-derived parameters demonstrated the highest correlation coefficients in predicting ECE and TTV (Table 17). The sextant set Gleason score best predicted pGS followed by the S12C set Gleason score. The greatest coefficient for predicting TTV for each of the biopsy sets was total cancer length (S12C>L6C>S6C). Percent tumor involvement, total cancer length, and number of positive cores in the S12C were better predictors of ECE than any biopsy parameter derived from the L6C or S6C sets. Collectively, the correlation analyses showed a superior association between S12C-derived parameters and both TTV and ECE when compared to S6C or L6C-derived parameters.

TABLE 16

| Characteristic | n = 178 |
|---|---|
| Median age (yrs.; interquartile range) | 62 (57-67) |
| Median PSA (ng./ml; interquartile range) | 5.8 (4.1-8.0) |
| Median free PSA (%; interquartile range) | 12.1 (7.9-16.3) |
| Abnormal DRE (%) | 24.7 |
| Median transitional zone volume (cc.; interquartile | 18.0 (12.0-31.0) |
| range) | |
| Median prostate volume (cc.; interquartile range) | 40.0 (30.0-57.0) |
| Median total tumor volume (cc.; interquartile range) | 0.56 (0.19-1.09) |
| Extracapsular extension (%) | 24.7 |
| Pathologic Gleason score (%) | |
| ≦6 | 47.8 |
| 7 | 46.6 |
| ≧8 | 5.6 |

TABLE 17

| | | Extracapsular | | | | | |
|---|---|---|---|---|---|---|---|
| | | extension | | Pathologic Gleason* | | Total tumor vol. | |
| | Biopsy set | (n = 178) | | (n = 178) | | (n = 136) | |
| | predictors | Coefficient | P Value | Coefficient | p Value | Coefficient | p Value |
| 12 core set | Gleason score | 0.334 | <0.001 | 0.493 | <0.001 | .323 | <0.001 |
| | No. positive cores | 0.447 | <0.001 | 0.271 | <0.001 | .536 | <0.001 |
| | Total Ca. length | 0.474 | <0.001 | 0.296 | <0.001 | .615 | <0.001 |
| | % tumor | 0.482 | <0.001 | 0.328 | <0.001 | .597 | <0.001 |
| | involvement | | | | | | |
| Sextant set | Gleason score | 0.428 | <0.001 | 0.596 | <0.001 | 0.350 | <0.001 |
| | No. positive cores | 0.333 | <0.001 | 0.178 | 0.018 | 0.416 | <0.001 |
| | Total Ca. length | 0.406 | <0.001 | 0.256 | 0.001 | 0.475 | <0.001 |
| | % tumor | 0.405 | <0.001 | 0.283 | <0.001 | 0.472 | <0.001 |
| | involvement | | | | | | |
| Lateral 6 | Gleason score | 0.276 | <0.001 | 0.405 | <0.001 | 0.229 | 0.019 |
| core set | No. positive cores | 0.343 | <0.001 | 0.246 | 0.001 | 0.498 | <0.001 |
| | Total Ca. length | 0.324 | <0.001 | 0.227 | 0.002 | 0.566 | <0.001 |
| | % tumor | 0.320 | <0.001 | 0.249 | 0.001 | 0.545 | <0.001 |
| | involvement | | | | | | |

*Pathologic Gleason score was categorized as <7 versus ≧7.

In multivariable analyses that controlled for biopsy parameters of the sextant and the L6C set, contributions from both the S6C and the L6C set were associated with TTV, ECE, and pGS 7 or greater (Table 18). The S6C Gleason score and number of positive lateral cores each had a greater than two-folds odds of predicting ECE. S6C Gleason score had twelve-fold odds ratio of predicting pGS, far greater than L6C (two-fold) or S6C (less than one-half-fold) number of positive cores. The S6C % tumor involvement and L6C total cancer length each independently predicted TTV.

Thirty-three (20.1%) of the patients in this study met Epstein's criteria (Epstein et al., 1994) for insignificant tumor. Using a test derived from the S6C parameters, 45 patients were incorrectly categorized as having insignificant cancer (Table 19). However, by adding the L6C parameters, only 10 patients were incorrectly categorized as having pathologic features indicative of insignificant cancer. Thus, the combination of S6C and L6C parameters increased the positive predictive value from 39% to 52% with only an 11% drop in the % negative predictive value. Alternatively, the S6C biopsy based test incorrectly categorized the significance of 49 (29.9%) tumors, as compared to the S12C based test which incorrectly categorized only 32 (19.5%) of tumors.

TABLE 18

| | Extracapsular | | | Pathologic | | | Total tumor | | |
|---|---|---|---|---|---|---|---|---|---|
| | extension | | | Gleason score | | | volume | | |
| | (n = 178) | | | (n = 178)* | | | (n = 136) | | |
| | Hazard | | p | Hazard | | p | Parameter | | |
| | Ratio | 95% CI | Value | Ratio | 95% CI | Value | Estimate | 95% CI | p Value |
| Sextant set | | | | | | | | | |
| Gleason score | 2.624 | 1.480-4.654 | 0.001 | 12.200 | 4.003-37.180 | <0.001 | | | 0.702 |
| No. Positive | | | 0.444 | 0.415 | 0.211-0.814 | 0.010 | | | 0.474 |
| cores | | | | | | | | | |
| Total cancer | | | 0.418 | | | 0.870 | | | 0.963 |
| length | | | | | | | | | |
| % Tumor | | | 0.090 | | | 0.057 | 0.066 | 0.037-0.095 | <0.001 |
| involvement | | | | | | | | | |
| Lateral 6 core set | | | | | | | | | |
| Gleason score | | | 0.978 | | | 0.169 | | | 0.749 |
| No. Positive | 2.283 | 1.375-3.791 | 0.001 | 2.071 | 1.082-3.962 | 0.028 | | | 0.627 |
| cores | | | | | | | | | |
| Total cancer | | | 0.178 | | | 0.582 | 0.005 | 0.001-0.009 | 0.022 |
| length | | | | | | | | | |
| % Tumor | | | 0.188 | | | 0.930 | | | 0.190 |
| involvement | | | | | | | | | |

*Pathologic Gleason score was categorized as <7 versus ≧7.

TABLE 19

| | | No. | No. non- | % Positive | % Negative | | |
|---|---|---|---|---|---|---|---|
| | | insignificant | insignificant | predictive | predictive | % | % |
| | | tumors (%) | tumors (%) | value | value | Sensitivity | Specificity |
| Sextant biopsy | | | | | | | |
| parameters | | | | | | | |
| Favorable | Sextant Gleason score <7 | 29 (17.7) | 45 (27.4) | 39 | | | |
| | and sextant Ca. | | | | | | |
| | involvement ≦4% | | | | | | |
| Unfavorable | Sextant Gleason score ≧7 | 4 (2.4) | 86 (52.4) | | 96 | 88 | 66 |
| | or sextant Ca. | | | | | | |
| | involvement >4% | | | | | | |
| Sextant and | | | | | | | |
| laterally directed | | | | | | | |
| biopsy | | | | | | | |
| parameters | | | | | | | |
| Favorable | Sextant Gleason score <7 | 11 (6.7) | 10 (6.1) | 52 | | | |
| | and sextant Ca. | | | | | | |
| | involvement ≦4% and ≦1 | | | | | | |
| | lateral positive core | | | | | | |
| | and total lateral Ca. | | | | | | |
| | length ≦3 mm | | | | | | |
| Unfavorable | Sextant Gleason score ≧7 | 22 (13.4) | 121 (73.8) | | 85 | 33 | 92 |
| | or sextant Ca. | | | | | | |
| | involvement >4% or >1 | | | | | | |
| | lateral positive core or | | | | | | |
| | total lateral Ca. length >3 mm | | | | | | |

Discussion

Variables closely associated with the outcome of interest underlie the development of nomograms with greater discriminatory ability and calibration. Building on previous work in this area (Sebo et al., 2000; Noguchi et al., 2001; Epstein et al., 1994; Grossklaus et al., 2002), it was determined whether the data in an extended field biopsy could enhance post-prostatectomy pathology prediction. It was hypothesized that the addition of the laterally directed biopsies to standard systematic sextant biopsy provides unique post-prostatectomy pathology predictive value. The analyses described herein demonstrated that the laterally directed biopsy cores contained unique information, improving the prediction of ECE, pGS, and TTV in prostatectomy specimens, in multivariable analyses that included biopsy information from the sextant set. This represents an advancement in the understanding of biopsy predictors of prostate pathology, and provides the rationale for incorporating extended field biopsy data in future prediction models and nomograms.

The study population represents a current cohort of patients with clinically localized prostate cancer detected with a S12C biopsy. While the superiority of S12C over sextant biopsy has been gaining acceptance, few studies have addressed the respective performance of various biopsy templates in predicting final pathologic parameters after radical prostatectomy. Taylor et al. (2002) reported recently that a greater number of significant cancers (defined as not <0.2 cc, organ confined, and pGS<7) are detected with an extended field biopsy. Sebo et al. (2000) recently reported that in prostate cancer patients diagnosed between March 1995 and April 1996 with an average of 6.2 cores, 20.8% had a tumor volume of less than 0.5 cc. In the present cohort, nearly one-half of the patients had a tumor volume of less than 0.5 cc, although some of these had a final GS of ≧7. The increase in the proportion of smaller tumors detected is likely due to the fact that the study population was biopsied with a systematic 12-core biopsy. Multiple authors have demonstrated continuing stage migration to smaller, less advanced tumors in more recently diagnosed patients cohorts. In addition, there may be an increased likelihood of detecting small tumors with the addition of laterally directed cores. The rate of ECE in our cohort was, however, only marginally less than that reported by Sebo et al. (2001) (24.7% versus 26.6%). The median age and PSA of the cohort compares similarly to recent reports in which patients have undergone a mean of 10 or more core biopsies (San Franasco et al., 2003; Presti et al., 2003). In aggregate, these data suggest that, on average, smaller tumors diagnosed with a S12C exhibit a similar proportion of features of aggressive cancer, as those diagnosed with sextant biopsy.

TTV, pGS, and ECE were chosen as outcome variables because they represent the best pathologic predictors for prostate cancer recurrence and indolence in patients without seminal vesicle invasion or lymph node involvement (Wheeler et al., 1998; Koch et al., 2000; Epstein et al., 1993). Over the last several years, various groups have suggested that the percent of cancer in the biopsy represents the best predictor of pathology findings after prostatectomy (Grossklaus et al., 2002; Sebo et al., 2001), whereas others have proposed that the number of positive cores (Wills et al., 1998) or the total mm of cancer in the biopsy specimen (Goto et al., 1998) best indicates prostate pathology. Mindful of these contradictory findings, it was elected to evaluate a broad range of biopsy predictors: number of positive cores, % of cancer involvement, total cancer length, and biopsy Gleason score. In designing this study, it was attempted to minimize the bias favoring the predictive potential of the L6C set. Therefore, patients with a history of biopsy prior to their S12C set were excluded, because many of these patients would have had a prior negative sextant biopsy.

In univariate correlation analyses, all the biopsy parameters from the S12C, S6C, and L6C set were significantly associated with TTV, ECE, and pathologic GS. Consistent with the hypothesis, the highest coefficients for predicting TTV and ECE were derived from the S12C set, suggesting that information contained in the S12C set is more representative of what is found in the prostatectomy specimen. Despite the superiority of the S12C, a significant correlation of the S6C with final pathologic parameters was found, consistent with previous studies based primarily on patients who had sextant biopsy. For example, Noguchi et al. (2001) demonstrated in a univariate analysis that the number of positive biopsy cores and total cancer length were significantly associated with cancer volume and the positive surgical margin rate. Sebo et al. (2000), analyzing 210 patients who underwent radical prostatectomy, found that the percent of tumor involvement and biopsy GS were significant predictors of pathologic stage.

It was further determined which of the biopsy-based parameters were independent predictors of prostate pathology in multivariable analyses. It was found that S6C and the L6C set both contributed significantly to the prediction of ECE, pGS (<7 vs. ≧7), and TTV. The significant S6C set biopsy parameters, which emerged in the multivariable analyses, were consistent with previous reports based on non-extended field biopsy schemes. Gilliland et al. (1999) reported that biopsy Gleason score independently predicted ECE status, a finding in congruence with the present S6C set Gleason score. pGS was best predicted by the S6C Gleason score with a greater than 12-fold odds. Interesting, an odds ratio of less than one-half was associated with the number of positive S6C cores in predicting pGS. This implies that if all else is kept equal, a greater number of positive sextant cores predicts a lower pathologic Gleason score. This finding could be explained by a greater sampling of the transition zone in the S6C than in the L6C set. Transitional zone tumors are less biologically aggressive and are generally associated with a lower Gleason score at the time of diagnosis (Mai et al., 2001) than peripheral zone tumors.

The L6C number of positive cores, notably, added a greater than two-fold odds in predicting ECE and pGS. The % tumor involvement of the S6C set predicted TTV, in agreement with the findings of Grossklaus et al. (2002) and Sebo et al. (2000). The L6C total cancer length contributed to the prediction of TTV independently of the S6C % tumor involvement. As compared to the original systematic sextant approach described by Hodge, the biopsy technique with laterally directed biopsies sampled more of the peripheral zone, an area more likely to harbor cancer. In particular, the S12C set included the highest cancer detection sites, such as the lateral apex and lateral base (Gore et al., 2001), likely resulting in a better assessment of the prostate tumor present.

Although there is clear evidence that a nomogram outperforms a stratifying risk model (Eastham et al., 2002), to gain preliminary insight into the value contained in the S12C set, a test was constructed for tumor insignificance based on Epstein's criteria (Epstein et al., 1994). It appears that addition of the laterally directed biopsy data to such a test improves its specificity and positive predictive value and decreases the incorrect categorization of tumor significance by 10.4%. This finding suggests that utilizing S12C based parameters would allow the physician to identify patients with insignificant tumor burden while minimizing the risk of under treating patients with significant tumors. One could potentially improve the robustness of a nomogram based on an extended field biopsy set with the addition of clinical and biomarker data.

Conclusion

The present study provides evidence that the total number of biopsy cores, and the location from which each core is obtained, greatly influences the accuracy of biopsy predictors of post-prostatectomy pathology. In the present cohort, both the S6C and L6C set independently contributed to the prediction of pathologic Gleason score, total tumor volume, and extracapsular extension. Pre-operative nomograms that utilize S12C data and specify biopsy parameters obtained from sextant and laterally directed biopsy cores will likely demonstrate improved performance in predicting post-prostatectomy pathology (e.g., indolent cancer or the presence of extracapsular extension).

### EXAMPLE 5

Validated cut-points for percent free PSA (% fPSA) and PSA density (PSAD) are based on cancer detection using primarily sextant biopsies. Systematic 12-core (S12C) biopsies that include standard sextant plus six laterally-directed biopsies significantly increase the detection rate for prostate cancer, and may detect a greater proportion of small volume cancers. PSA elevations that prompt biopsy in these patients, may be due to benign prostatic hyperplasia (BPH) rather than cancer.

Methods

This study evaluated 336 consecutive men whose PSA ranged between 4 and 10 (ng/ml) and who underwent a S12C biopsy. The medial 6-core biopsies (M6C) and the full S12C set comprise the study groups. Finger and ultrasound directed biopsy cores were excluded. ROC curves for PSATZD (PSA transition zone density), PSAD (PSA density), total PSA (tPSA), complexed PSA (cPSA), and % fPSA were constructed based on cancer diagnosis, and the AUCs were compared. In addition, the 90% sensitivities with their respective cut-points and specificities were calculated.

Results

The cancer detection rate was 37.7% and 28.4% for the S12C and M6C biopsy sets, respectively. Of note, for both biopsy study groups, PSATZD performed better than PSAD, which in turn performed better than % fPSA. The AUCs and 90% sensitivity values for the S12C and M6C groups are shown below.

TABLE 20

90% sensitivity

| | AUC | cutpoint | specificity |
|---|---|---|---|
| | S12C | | |
| PSATZD | 0.688 | 0.1000 | 0.131 |
| PSAD | 0.671 | 0.0634 | 0.165 |
| % fPSA | 0.600 | 23.05 | 0.16 |
| cPSA | 0.539 | 3.5996 | 0.117 |
| tPSA | 0.513 | 4.450 | 0.131 |
| | M6C | | |
| PSATZD | 0.719 | 0.1357 | 0.326 |
| PSAD | 0.696 | 0.0664 | 0.205 |
| % fPSA | 0.636 | 22.15 | 0.188 |
| cPSA | 0.548 | 3.5996 | 0.113 |
| tPSA | 0.511 | 4.450 | 0.13 |

The performance of the three serum tests with the greatest AUC, PSATZD, PSAD, and % FPSA, appears to be degraded with a S12C biopsy compared to the traditional sextant biopsy.

### EXAMPLE 6

To examine the predictors of prostate cancer on a second systematic 12-core biopsy (S12C) in patients with an initial S12C without evidence of prostate cancer, the study evaluated 1,047 consecutive patients who underwent an initial S12C biopsy. 144 of these patients had a S12C without evidence of prostate cancer and underwent a repeat S12C biopsy. Of these patients, 95 had a prostate serum antigen (PSA) at initial biopsy between 2.5 and 10 ng/ml and ultimately comprised the study population. Parameters that were evaluated included initial and repeat biopsy PSA, initial and repeat percent free PSA (% fPSA), initial and repeat biopsy digital rectal exam (DRE) status (normal versus abnormal), presence of high grade prostatic intraepithelial neoplasia (PIN) on initial biopsy, presence of atypical small acinar proliferation (ASAP) on initial biopsy, poor DRE change (initial normal→repeat abnormal), PSA doubling-time (PSAdt=log(2)*(number of days between PSA measurement)/[log(repeat PSA)−log(initial PSA)]), and yearly inter-biopsy PSA changes (yibPSA=[(repeat PSA)−(initial PSA)]/(number of days between PSA measurement)*365). Statistical methods included the Mann-Whitney U test, Pearson Chi-Square test, and multivariable logistic regression analysis.

Results

In univariable analyses PSAdt, yibPSA, initial and repeat PSA, initial and repeat % fPSA, poor DRE change, repeat DRE status, and presence of ASAP were not significant predictors of prostate cancer at repeat biopsy. However, both initial DRE status (P=0.034) and the presence of PIN (P=0.010) were significant predictors of prostate cancer at repeat biopsy. In multivariable logistic regression analysis, only the presence of PIN remained a significant predictor of prostate cancer (P=0.012).

Conclusions

The results suggest that for patients with a PSA between 2.5 and 10 ng/ml whose initial S12C biopsy contains PIN but not cancer, the presence of PIN alone is an indication to re-biopsy.

### EXAMPLE 7

To determine whether data obtained through biopsy can be used to help predict side-specific posterolateral ECE, and whether a systematic, 12-core biopsy regimen (S12C) outperforms a S6C, 181 consecutive patients who underwent a S12C followed by radical retropbital prostatectomy (RRP) were analyzed. RRP specimens were processed using the whole-mount method. PSA, DRE, maximum biopsy Gleason Grade (mGG), number of positive cores (PC), number of contiguous positive cores (CPC) and percent of the biopsy material with cancer (% CA) were tested for their ability to predict posterolateral ECE using multivariate logistic regression analysis, and the Pearson Chi-Square test.

Results

The majority of the patients in the dataset with posterolateral ECE, had this as the only adverse pathologic feature of their prostate cancer. Only 19% (95% CI=1-33%) also had positive lymph nodes SVI, or ECE at the bladder neck or apex. Only 8% (CI=2-25%) had additional adverse pathological features when limited to those with a PSA≦10 ng/ml and biopsy GS≦7. Although in multivariate analyses controlling for DRE and mGG, the number of PC, % CA, and the number of CPC in the sextant cores were all predictors of ECE, on addition of the corresponding parameters from S12C data, these predictors were no longer significant, indicating that for each of the three parameters, S12C data was superior to sextant core data. The AUC of 12CR % CA was 0.88 (95% CI=0.82-93). S12C CPC and number of PC had sensitivities and specificities comparable to % CA.

Thus, data obtained through a S12C biopsy were independent predictors of posterolateral ECE and were superior to analogous sextant biopsy data.

### EXAMPLE 8

To develop a nomogram to predict the side of ECE in RP, 763 patients with clinical stage Tlc-T3 prostate cancer who were diagnosed with a systematic biopsy and were subsequently treated with RP were studied. A ROC analyses were performed to assess the predictive values of each variable alone and in combination. The variables included an abnormality on DRE, the worst Gleason score (worst Gleason score in any one core), number of cores with cancer, percent cancer in a biopsy specimen (PERCA) on each side and serum PSA level.

Results

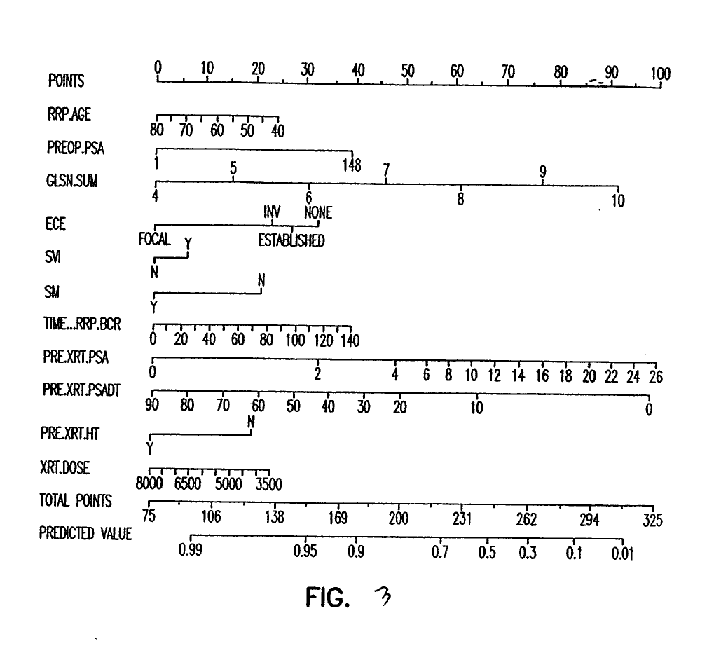

Overall, 31% of the patients had ECE and 17% of the 1526 sides of the prostate had ECE. Of the 812 sides with no palpable abnormality on DRE, 95 (11.5%) had ECE at the ipsilateral side compared to 20 (58.8%) of 34 sides with T3 nodule. Of the 500 sides with no cancer in a biopsy (recorded as Gleason sum 0), 30 (6%) had ECE at the ipsilateral side compared to 64 (52.4%) of 122 sides with Gleason sum 7 (4+3) 10 cancers. The area under the curve (AVC) of DRE, biopsy Gleason sum and PSA in predicting the side of ECE was 0.648, 0.724 and 0.627, respectively, and was 0.763 when these parameters were combined. Further, this was enhanced by adding the information of systematic biopsy with the highest value of 0.787 with a percent cancer. Based on the regression analysis, the nomogram was constructed (FIG. 2) and the accuracy of this nomogram was confirmed by the internal calibration.

Conclusions

A nomogram incorporating pre-treatment variables on each side of the prostate can provide accurate prediction of the side of ECE in RP specimens. Thus, this nomogram can assist the clinical decision such as resection or preservation of neurovascular bundle prior to radical prostatectomy.

### EXAMPLE 9

To develop a nomogram to improve the accuracy of predicting the freedom from PSA progression after salvage external beam radiotherapy (XRT) for biochemical recurrence (BCR) following radical prostatectomy (RP), pre- and post-prostatectomy clinical-pathological data and disease follow-up for 375 patients receiving salvage XRT was modeled using Cox proportional hazards regression analysis. Indications for salvage XRT included persistently elevated PSA following prostatectomy (n=108) and BCR (PSA>0.1, N=267) with or without clinically evident LR (local recurrence). Biochemical progression after salvage XRT was defined as two consecutive PSA rises greater than 0.1. Pre-radiotherapy variables were selected for use in the nomogram. These included pre-operative PSA, pre-XRT PSA, pre-XRT PSA doubling time, Gleason sum, pathological stage, surgical margins status, time from RP-to-BCR, neoadjuvant hormonal therapy and XRT dose.

Results

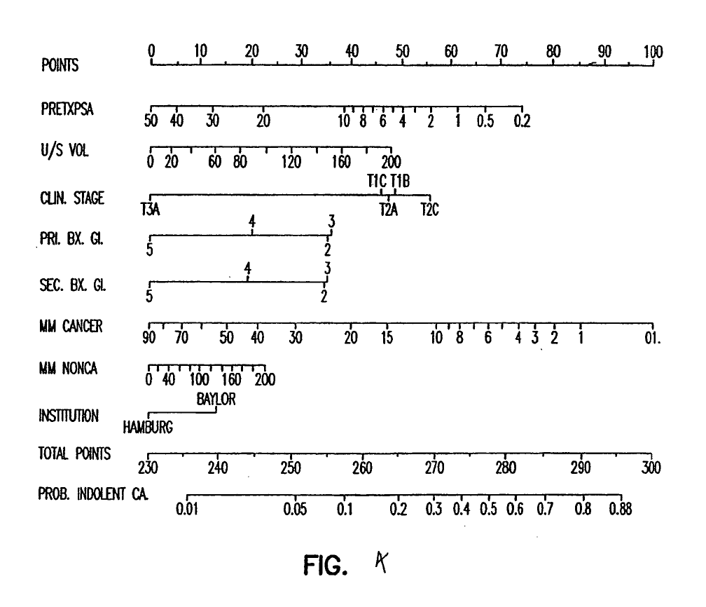

The median follow-up after XRT was 35.8 months. Overall, the 2-year and 5-year actuarial progression-free probability (PFP) after salvage XRT was 57% and 31% respectively. The median freedom from progression was 32.2 months. The median time-to-recurrence after XRT was 11.6 months. Multivariate Cox regression analysis revealed Gleason sum (HR 13.9, P=0.0002), pre-XRT PSA (HR 2.2, P=0.001), PSA doubling time (HR 0.45, P=0.002), positive surgical margins (HR 0.54, P=0.003) and neoadjuvant hormone therapy (HR 0.54, P=0.003) as significant prognostic variables. A nomogram to predict the 2-year progression-free probability was generated using all pre-selected variables (FIG. 3). The nomogram had a bootstrap-corrected concordance index of 0.73.

Given the morbidity and that a minority of patients derived a durable benefit from salvage radiotherapy in this cohort, it is evidence that patient selection is critical when considering this therapy. This nomogram is a tool to aid in identifying the most appropriate patients to receive salvage radiotherapy. The nomogram predicts a 2-year PFP between 65-95% for a typical patient with a pre-XRT PSA<2 ng/mL, PSADT>10 months, Gleason sum 2-7 and pT3a prostate cancer following salvage radiotherapy.

### EXAMPLE 10

To determine whether the transition zone volume (TZV) and total prostate volume (TPV) are independent predictors of PSA, 560 men who underwent a systematic 12-core biopsy performed under ultrasound guidance were analyzed, among a multi-racial population with and without positive prostate biopsies from total population (n=1047) of men who in a retrospective cohort study. Entry criteria were collection and analysis of pre-biopsy serum for determination of total and free serum PSA. TZV and TPV were calculated using the standard elliptical formula=height×width×length×0. 524. Multivariable logistic and multivariate linear regression analyses were used to determine if race, age, TZV, and TPV were independent predictors and risk factors of total PSA, free PSA and highest quartile of total PSA.

Results

Of the 560 men in the cohort, 80%, were Caucasian, 4% were African-American, 5.2% Hispanic 9% Asian, and 14.8% were of mixed or “other” designations.

TABLE 21

| Variables in | | | | Variables in | | | |
|---|---|---|---|---|---|---|---|
| Logistic | | | | Logistic | | | |
| Regression | | Odds | Confidence | Regression | | Odds | Confidence |
| Analysis | p value | Ratio | Interval | Analysis | p value | Ratio | Interval |
| Race | 0.2667 | 1.097 | 0.93-1.29 | Race | 0.2667 | 1.084 | 0.92-1.28 |
| Age | 0.0036 | 1.054 | 1.02-1.09 | Age | 0.0036 | 1.048 | 1.01-1.09 |
| Biopsy Status | 0.0200 | 1.981 | 1.11-3.52 | Biopsy Status | 0.0200 | 2.143 | 1.19-3.85 |
| High TZV | 0.0003 | 3.06 | 1.74-5.64 | High TPV | <0.0001 | 4.148 | 2.26-7.63 |

When controlling for race, age and biopsy status using linear regression analysis, TZV and TPV are each separately significant predictors of PSA (P<0.0001 each) among men with either positive or negative systematic 12-core biopsies. Race did not prove to be an independent predictor of PSA in this study population.

### EXAMPLE 11

Men diagnosed with clinically localized prostate cancer have a number of treatment options available, including watchful waiting, radical prostatectomy and radiation therapy. With the widespread use of serum PSA testing, prostate cancers are being diagnosed at an earlier point in their natural history, with many tumors being small and of little health risk to the patient, at least in the short-term. To better counsel men diagnosed with prostate cancer, a statistical model that accurately predicts the presence of cancer based on clinical variables (serum PSA, clinical stage, prostate biopsy Gleason grade, and ultrasound volume), and variables derived from the analysis of systematic biopsies, was developed.

Materials and Methods

The analysis included 1,022 patients diagnosed through systematic needle biopsy with clinical stages Tlc to T3 NO or NX, and MO or MX prostate cancer who were treated solely with radical prostatectomy at one of two institutions. Additional biopsy features included number and percentage of biopsy cores involved with cancer and highgrade cancer, in addition to total length of biopsy cores involved. Indolent cancer was defined as pathologically organ confined cancer, ≦0.5 cc in volume, and without poorly differentiated elements. Logistic regression was used to construct several prediction models and the resulting nomograms.

Results

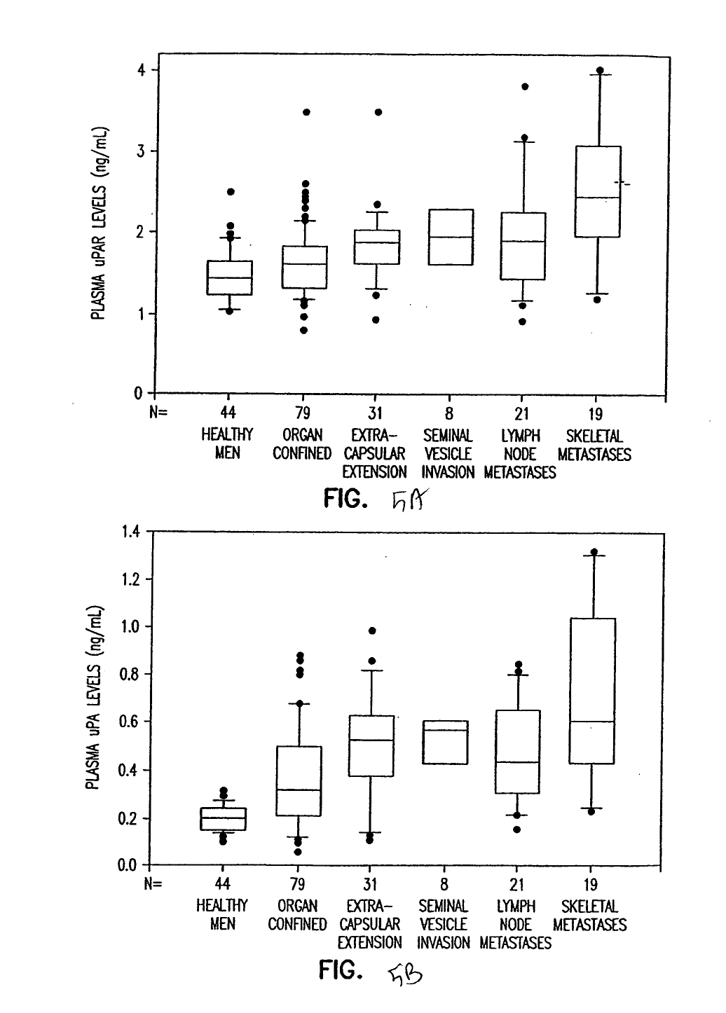

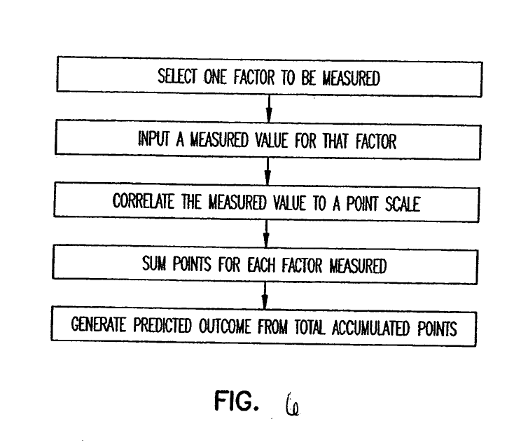

Overall, 105 (10%) of the patients had indolent cancer. The nomogram (FIG. 4) predicted the presence of an indolent cancer with discrimination (area under the receiver operating characteristic curves) for various models ranging from 0.82 to 0.90. Calibration of the models appeared good.

Conclusions

Nomograms incorporating pre-treatment variables (clinical stage, Gleason grade, PSA, and the amount of cancer in a systematic biopsy specimen) can predict the probability that a man with prostate cancer has an indolent tumor. These nomograms have excellent discriminatory ability and good calibration and may benefit both patient and clinician when the various treatment options for prostate cancer are being considered.

### EXAMPLE 12

To assess the prognostic significance of the sites of +SM in RP specimens, 1368 consecutive patients who were treated with RP by 2 surgeons were studied. Detailed pathologic features of cancer were assessed by one pathologist. The adjuvant radiation therapy before PSA recurrence was assessed as a time-dependent covariate to analyze PSA progression free probability (PFP). Median follow-up was 48 months.

Results

Overall, 179 patients (13%) had +SM. Of the 169 patients with the detailed results of +SM sites, 122 (73%) had only single +SM site, 32 (19%) had 2 sites and 14 (8%) had >2+SM sites. PFP at 5 year for patients with a single or 2+SM sites was 71% and 74%, significantly better than 36% of patients with >2+SM sites (p=0.006 and p=0.02, respectively). Of a total of 246+SM sites, 30% were in the apical shave sections 29% in the apex (first two whole mount step sections), 24% in the mid, 9% in base section (last two sections), 6% in bladder neck, and 2% over seminal vesicles. In the analysis of the transverse section, 24% were in the anterior, 19% in the postero-lateral 14% in the posterior, 5% in the lateral. PFPs at 5 years for patients with a single +SM in the apical was 69% and in the apex, 84%, significantly better than 27% with a single +SM at the base (p=0.008 and p=0.01, respectively) while the patients with +SM in mid or bladder neck had an intermediate PFPs. More cancers were confined to the prostate when the +SM was at the apical (83%) or apex (74%) than at the base (14%). PFPs at 5 years for patients with a single +SM in the posterior was 48%, significantly worse than 79% of the patients with +SM in the anterior (p=0.033). In a Cox hazard regression analysis for the various models, +SM in the apical was only significant predictor of PSA progression (p=0.0021) when other established pathological features and serum PSA level were controlled. The +SM rate significantly decreased over the time as did the number of sites of +SM per prostate (p<0.005). Also the proportion of all +SM that were apical or apex significantly increased (p<0.005).

Conclusions

Prognostic significance of +SM may depend on the location of +SM in RP specimens. Although patients with +SM in the base and/or in the posterior had a worse PFP than other +SM locations, +SM in the apical shave sections, which has been significantly increasing, was the only significant predictor in a multivariate analysis. Thus, more attention should be paid for +SM in apical sections.

### EXAMPLE 13

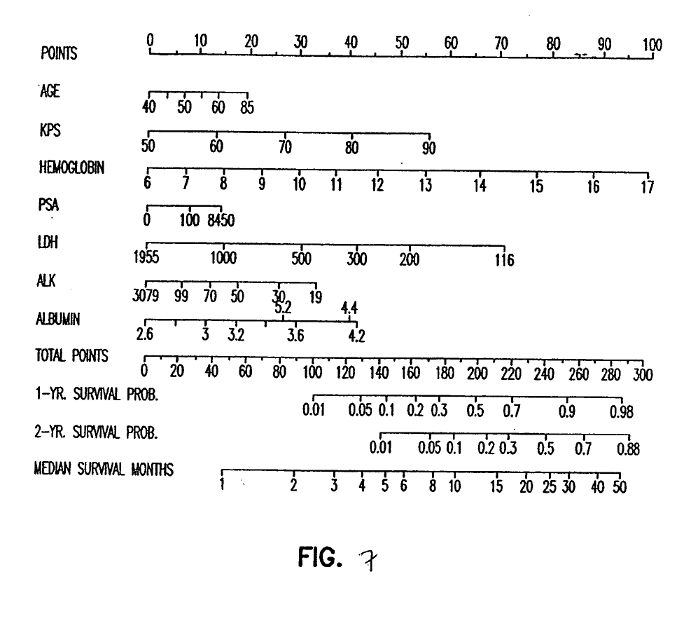

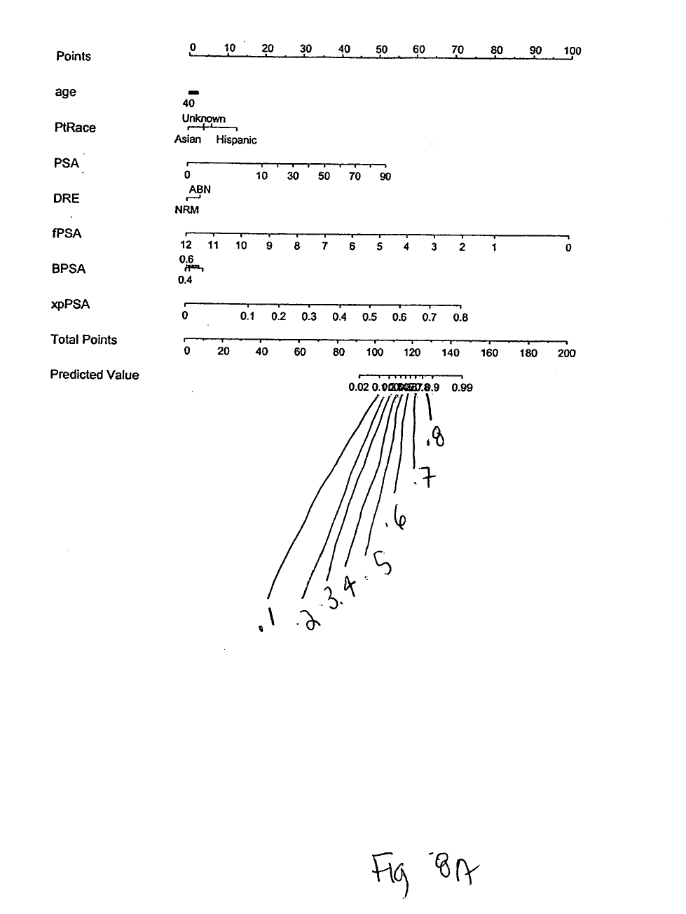

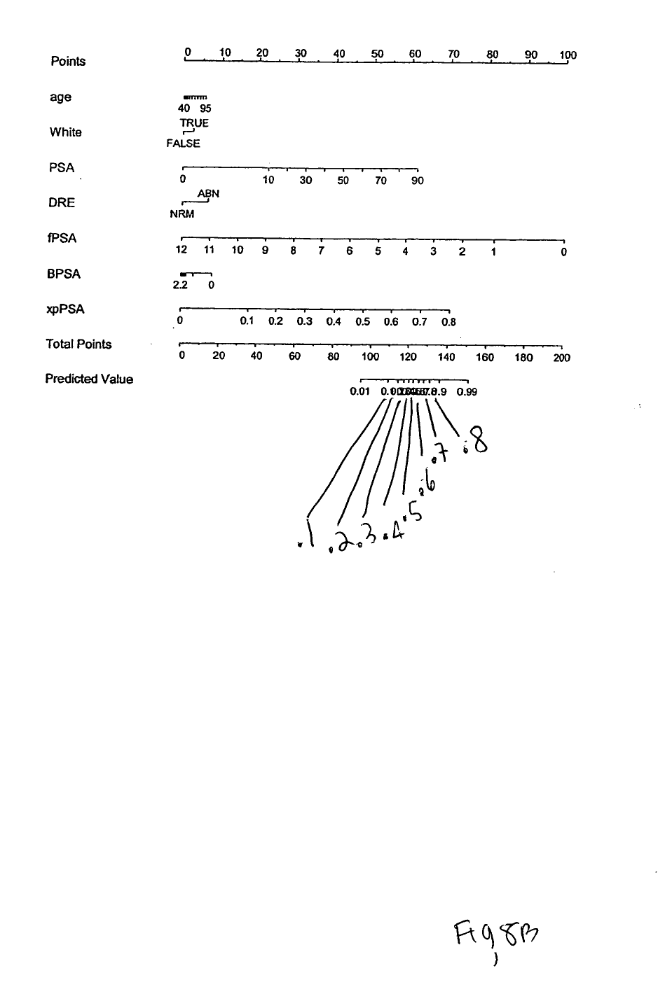

The urokinase plasminogen activation cascade has been closely associated with poor clinical outcomes in a variety of cancers. The following hypothesis was tested: that pre-operative plasma levels of the major components of the urokinase plasminogen activation cascade (urokinase plasminogen activator, UPA; the UPA receptor, UPAR; and the inhibitor, PAI-1) would predict cancer presence, stage, and disease progression in patients undergoing radical prostatectomy (FIG. 5).

Plasma levels of UPA, UPAR, and PAI-1 were measured pre-operatively in 120 consecutive patients who underwent radical prostatectomy for clinically localized disease and post-operatively in 51 of these patients. Marker levels were measured in 44 healthy men, in 19 patients with metastases to regional lymph nodes, and in 10 patients with bone metastases.

UPA and UPAR levels but not PAI-1 levels were elevated in prostate cancer patients compared with healthy subjects (P=0.006 and P=0.021, respectively) and were highest in patients with bone metastases. Elevated UPA and UPAR levels were associated with extraprostatic disease (P=0.046 and P=0.050, respectively) and seminal vesical involvement (P=0.041 and P=0.048, respectively). Elevated UPA and UPAR levels were correlated with prostatic tumor volume (P=0.036 and P=0.030, respectively). In multivariate analysis, pre-operative plasma UPA and UPAR levels, as well as biopsy Gleason sum, were independent predictors of prostate cancer progression (P=0.034, P=0.040, and P=0.048, respectively). In patients with disease progression, pre-operative plasma UPA and UPAR levels were higher in those with features of aggressive than in those with features of non-aggressive failure (P=0.050 and P=0.031, respectively).

While plasma UPA and UPAR levels were elevated in men with prostate cancer compared to healthy men, they were most dramatically elevated in men with bony metastases. Pre-operative plasma levels of UPA and UPAR levels were associated with established features of biologically aggressive prostate cancer and disease progression. In multivariate analysis, pre-operative UPA and UPAR levels were independent predictors of disease progression in men undergoing radical prostatectomy. In combination with other clinical and pathologic parameters, plasma UPA and UPAR levels may be useful in selecting patients to enroll in clinical neo-adjuvant and adjuvant therapy trials.

### EXAMPLE 14

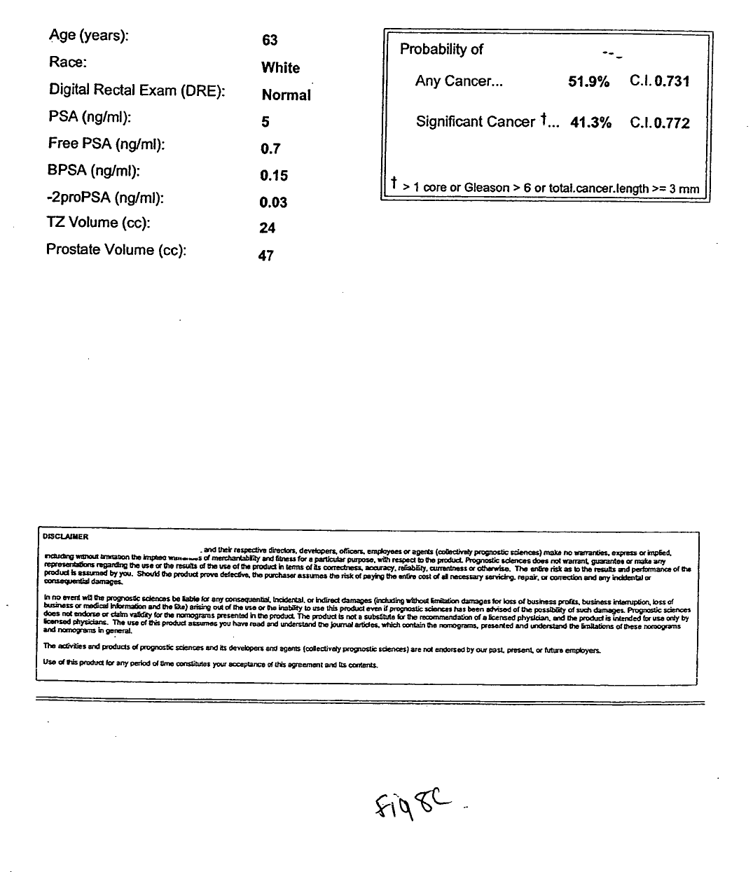

To provide a nomogram useful to predict progression to death in patients with metastases at the time of primary or subsequent therapy, serum markers may be employed with factors such as Karnofsky performance status, hemoglobin, PSA, lactate dehydrogenase, alkaline phosphatase and albumin to predict time to death including median, 1 year and 2 year survival (FIG. 7). In one embodiment, the nomogram is employed to predict time to death in patients with hormone sensitive prostate cancer. In another embodiment, the nomogram is employed to predict time to death in patients with hormone refractory disease. In one embodiment, one or more of TGF-β₁, IL6sR, IL6, VEGF, sVCAM, UPA or UPAR levels or amounts are employed with Karnofsky performance status, hemoglobin, PSA, lactate dehydrogenase, alkaline phosphatase and albumin. In another embodiment, one or more of TGF-β₁, IL6sR, IL6, VEGF, sVCAM, UPA or UPAR levels or amounts are employed in place of one or more of Karnofsky performance status, hemoglobin, PSA, lactate dehydrogenase, alkaline phosphatase and albumin.

### EXAMPLE 15

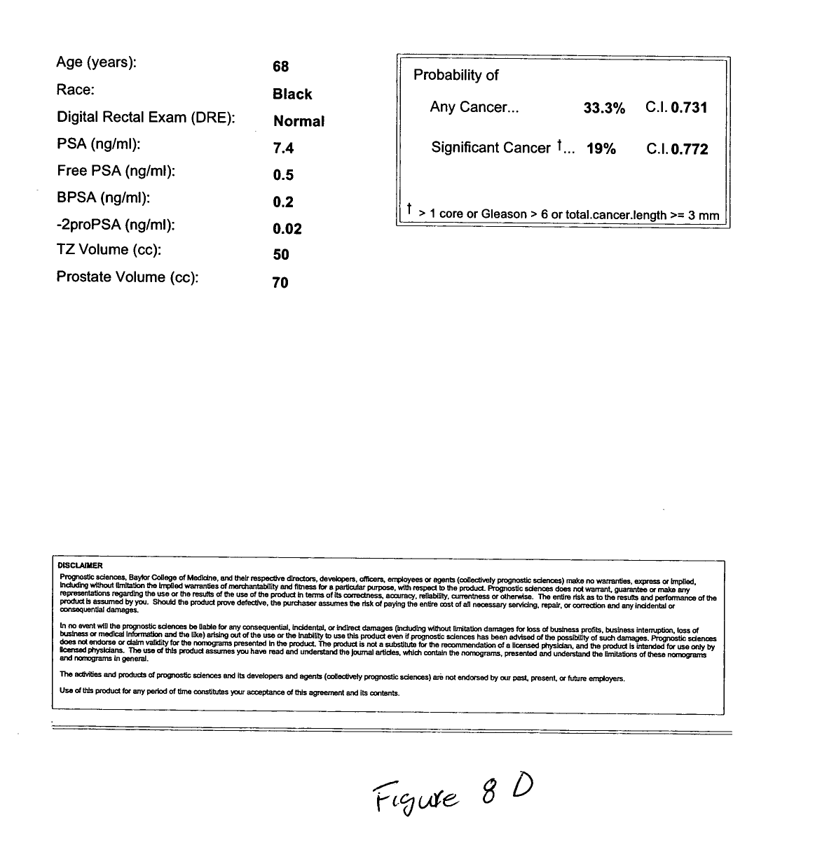

FIG. 8 provides nomograms useful to predict the risk of prostate cancer (FIG. 8A), including a prediction of significant prostate cancer (FIG. 8B).

### BIBLIOGRAPHY

Adams et al., Cancer Res., 60:2898 (2000).
Benjamin et al., J. Clin. Invest., 103:159 (1999).
Breiman et al., Classification and Regression Trees. Monterey, Calif., Wadsworth and Brooks/Cole (1984).
Chang et al., J. Urol., 160:2111 (1998).
Cowen et al., Med. Decis. Making, 18:376 (1998).
Cox, J. Royal Statistical Society, B34, 187-220 (1972)
Cristianni et al., An Introduction to Support Vector Machines,
Cambridge University Press (2000).
Cupp et al., J. Urol., 153:1543 (1995).
D'Amico et al., J. Clin. Oncol., 17:168 (1999).
D'Amico et al., J. Clin. Oncol., 18:1164 (2000).
D'Amico et al., JAMA, 280:969 (1998).
Danielpour et al., Annals New York Academy of Sciences, pp. 300-302 (1990).
Danielpour et al., J. Cell. Phy., 138:79 (1989).
Danielpour, J. Immunol. Meth., 158: 17 (1993).
Dasch et al., Annals New York Academy of Sciences, pp. 303-305 (1990).
Dorey et al., Stats. in Med., 12:1589 (1993).
Eastham et al., In: NJ Vogelzang, PT Scardino, WU Shipley, and DS Coffey (eds), Comprehensive textbook of Genitourinary Oncology, pp 722-738 (2000).
Eastham et al., Semin. Urol. Oncol., 20:108 (2002).
Efron B, Tibshirani R J. An introduction to the bootstrap. New York: Chapman & Hall; 1993.
Egawa et al., Urology, 52:433 (1998).
Engeler et al., Urology, 40:346 (1992).
Epstein et al., J. Urol., 149:1478 (1993).
Epstein et al., J. Urol., 160:2407 (1998).
Epstein et al., J. Urol., 160:97 (1998).
Epstein et al., JAMA, 271:368 (1994).
Flanders, J. Cell Biol., 108:653 (1989).
Gilliland et al., J. Urol., 162:1341 (1999).
Gore et al., J. Urol., 165:1554 (2001).
Goto et al., Int. J. Urol., 5:337 (1998).
Graefen et al., J. Clin. Oncol., 20:3206 (2002).
Graefen et al., Urologic Oncology, 7:141 (2002).
Greene et al., Br. J. Urol., 68:499 (1991).
Greene et al., Campbell's Urology, vol. 1, 6th ed, W. B. Saunders Co. (1992).
Greene et al., J. Urol., 146:1069 (1991).
Grossklaus et al., J. Urol., 167:2032 (2002).
Harrell et al., JAMA, 247:2543 (1982).
Harrell et al., Stat. Med., 15:361 (1996).
Harrell, In: Regression Modeling Strategies with Applications to Linear Models, Logistic Regression, and Survival Analysis, Springer-Verlag, NY (2001).
Hastie, The Elements of Statistical Learning, Springer (2001).
Hobisch et al., J. Pathol., 191:239 (2000).
Hodge et al., J. Urol., 142:71 (1989).
Kattan et al., Cancer, 79:528 (1997).
Kattan et al., J. Clin. Oncol., 18:3352 (2000).
Kattan et al., Int. J. Radiol. Oncol. Biol. Phys., 48:1469 (2000).
Kattan et al., J. Gen. Intern. Med., 12:299 (1997).
Kattan et al., Urology, 58:393 (2001).
Kattan et al., J. Clin. Oncol., 17:1499 (1999).
Kattan, J. Natl. Cancer Inst., 90:766 (1998).
Koch et al., J. Urol., 164:749 (2000).
Lilja et al., Clin. Chem., 37:1618 (1991).
Lucas et al., J. Immunol., 145:1415 (1990).
Mai et al., Urol. Int., 66:191 (2001).
Maru et al., J. Urol., 161:1280 (1999).
Merrill et al., J. Urol., 163:503 (2000).
Mikolajczyk, et al., Cancer Research, 60:756 (2000).
Miller, Breast Cancer Res. Treat, 75:S45 (2002).
Nakashima et al., Clin. Cancer Res., 6:2702 (2000).
Narain et al., Prostate, 49:185 (2001).
Newcomer et al., J. Urol., 158:1427 (1997).
Noguchi et al., J. Urol., 164:1596 (2000).
Noguchi et al., J. Urol., 166:104 (2001).
Oesterling et al., JAMA, 269:57 (1993).
Ohori et al., Am. J. Surg. Pathol., 17:1252 (1993).
Ohori et al., Cancer, 74:104 (1994).
Ohori et al., Prostate, 23:271 (1993).
Partin et al., JAMA, 277:1445 (1997).
Partin et al., JAMA, 278:118 (1997).
Partin et al., Urol. Clin. North Am., 20:713 (1994).
Partin et al., Urology, 58:843 (2001).
Pisany et al., Int. J. Radial. Oncol. Biophys., 36:585 (1996).
Pound et al., JAMA, 281:1591 (1999).
Presti et al., J. Urol., 169:125 (2003).
Rosen et al., J. Urol., 148:331 (1992).
Rumelhart et al., (eds), Parallel Distributed Processing: Exploration in the Microstructure of Cognition Volume 1, Foundations Cambridge, Mass., The MIT Press (1986).
San Francisco et al., J. Urol., 169:136 (2003).
Sebo et al., Cancer, 91:2196 (2001).
Sebo et al., J. Urol., 163:174 (2000).
Shariat et al., J. Clin. Oncol., 19:2856 (2001c).
Shariat et al., J. Clin. Oncol., 20:833 (2002).
Shariat et al., Urology, 58:1008 (2001b).
Shipley et al., JAMA, 281:1598 (1995).
Smaletz et al., J. Clin. Oncol., 20:3972 (2002).
Spence et al., Int. J. Biol. Markers, 17:119 (2002).
Sridhara et al., J. Clin. Oncol., 13:2944 (1995).
Stamey et al., J. Urol., 139:1235 (1998).
Stamey et al., J. Urol., 167:103 (2002).
Stamey, Clin. Chem., 47:631 (2001).
Stapleton et al., Cancer, 82:168 (1998).
Stein et al., J. Urol., 147:942 (1992).
Stenman et al., Cancer Res., 51:222 (1991).
Taylor et al., Urology, 60:841 (2002).
Thompson et al., J. Cell Biol., 108:661 (1989).
Wei et al., J. Clin. One., 20:557 (2002).
Wheeler et al., Hum. Pathol., 29:856 (1998).
Wheeler et al., Prostate, 25:274 (1994).
Wills et al., Urology, 51:759 (1998).
Yang et al., J. Urol., 159:941 (1998)
Zagars et al., Cancer, 79:1370 (1997).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A nomogram for the graphic representation of a quantitative risk or probability of prostate cancer in a patient, comprising: a plurality of scales and a solid support, the plurality of scales being disposed on the support and comprising a scale for a plurality factors including two or more of age, race, DRE, PSA level, free PSA level, BPSA level, and/or proPSA level, a points scale, a total points scale and a predictor scale, wherein the scales for age, race, DRE, PSA level, free PSA level, BPSA level, and/or proPSA level each has values on the scales, and wherein the scales for age, race, DRE, PSA level, free PSA level, BPSA level, and/or proPSA level are disposed on the solid support with respect to the points scale so that each of the values on age, race, DRE, PSA level, free PSA level, BPSA level, and/or proPSA level can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, race, DRE, PSA level, free PSA level, BPSA level, and/or proPSA level can be added together to yield a total points value, and the total points value can be correlated with the predictor scale to predict the risk of or quantitative probability of prostate cancer.

2. The nomogram of claim 1 wherein the solid support is a laminated card.

3. The nomogram of claim 1 wherein the risk or quantitative probability of significant prostate cancer is predicted.

4. The nomogram of claim 1 wherein the factors include free PSA level, proPSA level and PSA level.

5. A method to predict prostate cancer and/or significant prostate cancer in a patient comprising: providing a value for a set of factors for a patient, which factors include two or more of age, race, DRE, PSA level, free PSA level, BPSA level, and/or proPSA level; matching the factors to the values on the scales of the nomogram of claim 1; determining a separate point value for each of the factors; adding the separate point values together to yield a total points value; and correlating the total points value with a value on the predictor scale of the nomogram to predict the risk or probability of prostate cancer in the patient.

6. An apparatus for predicting the risk or probability of prostate cancer, which apparatus comprises:
a) a correlation of a set of factors for each of a plurality of persons previously diagnosed with prostate cancer with the incidence of prostate cancer for each person of the plurality of persons, wherein the set of factors comprises a plurality of factors including two or more of age, race, DRE, PSA level, free PSA level, BPSA level, and/or proPSA level; and
b) a means for comparing an identical set of factors determined from a patient to the correlation to predict the risk or quantitative probability of prostate cancer.

7. An apparatus, comprising:
a data input means, for input of information for a plurality of patient factors, factors including two or more of age, race, DRE, PSA level, free PSA level, BPSA level, and/or proPSA level;
a processor, executing a software for analysis of the information;
wherein the software analyzes the information and provides the risk or probability of prostate cancer in the patient.

8. The apparatus of claim 7 wherein the plurality of factors are input manually using the data input means.

9. The apparatus of claim 7 wherein the software constructs a database of the information.

10. A method to determine the risk or probability of prostate cancer in a patient, comprising:
a) providing a value for a plurality of patient factors, factors including two or more of age, race, DRE, PSA level, free PSA level, BPSA level, and/or proPSA level; and
b) correlating the values for the plurality of factors with the risk or probability of prostate cancer in the patient.

11. The method of claim 10 wherein the values are correlated to the risk of significant prostate cancer in the patient.

12. The method of claim 10 wherein the values for three or more of the factors are provided.

13. The method of claim 10 wherein the values for four or more of the factors are provided.

14. The method of claim 10 wherein the correlating is conducted by a computer.

15. The method of claim 10 wherein the proPSA level is the −2 proPSA level.

16. A method to determine the risk or probability of a prostate cancer in a patient, comprising:
a) inputting information to a data input means, wherein the information comprises values for a plurality of patient factors including two or more of age, race, DRE, PSA level, free PSA level, BPSA level, and/or proPSA level;
b) executing a software for analysis of the information; and
c) analyzing the information so as to provide the risk or probability of prostate cancer in the patient.

17. The apparatus of claim 6 or 7 wherein the proPSA is the −2proPSA isoform.

18. The method of claim 5, 10 or 16 wherein the factors include free PSA and proPSA.

19. The apparatus of claim 6 or 7 wherein the factors include free PSA and proPSA.
